(19) United States
(12) Patent Application Publication
(10) Pub. No.: US 2026/0264227 A1
(43) Pub. Date: Sep. 10, 2026

(54) PROSTHETIC LIMB APPARATUS AND METHODS

(71) Applicant: Atom Limbs Inc., Palo Alto, CA (US)

(72) Inventors: Erik J. SHAHOIAN, Sonoma, CA (US); Steven SAVAS, San Carlos, CA (US); Joshua R. FUNAMURA, San Carlos, CA (US); Eric M. MONSEF, Monte Sereno, CA (US); Doug SATZGER, Menlo Park, CA (US); Tyler HAYES, San Francisco, CA (US)

(21) Appl. No.: 18/873,726

(22) PCT Filed: Jun. 12, 2023
(86) PCT No.: PCT/US2023/068313

§ 371 Date: Dec. 10, 2024

Related U.S. Application Data
(60) Provisional application No. 63/351,336, filed on Jun. 10, 2022.

(51) Int. Cl. B25J 9/16; A61F 2/58; B25J 9/10; B25J 13/08; B25J 15/00

(52) U.S. Cl. CPC B25J 9/1612; A61F 2/586; B25J 9/102; B25J 13/085; B25J 13/088; B25J 15/0009

(57) ABSTRACT

Methods and apparatuses (devices, systems, etc.) for artificial limbs, including prosthetics and/or robotic arms. The methods and apparatuses described herein may be used as part of a powered prosthetic device to be worn by a user or they may be part of a robotic apparatus that may be operated remotely or automatically, even in the absence of a human operator.

### CLAIM OF PRIORITY

This patent application claims priority to U.S. provisional patent application No. 63/351,336, titled “PROSTHETIC LIMB APPARATUS AND METHODS”, and filed on Jun. 10, 2022, herein incorporate by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Hand and arm amputees benefit greatly from prosthetic replacements. Prosthetic hands, wrists, and/or arms restore lost functionality and provide independence to users. However, existing solutions have deficient control and do little to restore functionality. For instance, existing solutions cause responsive delay with joint movement, including wrist rotation that detracts from more closely mimicking the functionality of a natural appendage. As another example, existing solutions insufficiently synchronize prosthetic movements.

Although powered (e.g., robotic) prosthetic devices have been proposed, these apparatuses have bene poorly adopted, as they have proven difficult to control and operated, and are also uncomfortable, as they may be heavy. Improved prosthetic apparatuses are therefore desirable.

### SUMMARY OF THE DISCLOSURE

Described herein are methods and apparatuses (devices, systems, etc.) related to artificial limbs, including prosthetics and/or robotic arms. These methods and apparatuses described herein may be used as part of a powered prosthetic device to be worn by a user or they may be part of a robotic apparatus that may be operated remotely or automatically, even in the absence of a human operator.

In general, these apparatuses may include any of the features or components described herein in any combination or individually. For example, described herein are elbow joint assemblies (e.g., robotic elbow joint assemblies), wrist assemblies (e.g., robotic wrist assemblies) and hand/finger assemblies (including robotic finger assemblies). An apparatus may include all or some of these features (e.g., a robotic elbow joint assembly, robotic wrist assembly, robotic finger assembly, etc.). These apparatuses may include one or more processors for controlling operation of these apparatuses. These apparatuses may generally include a power supply (rechargeable power supply, wall power adapter, etc.), and may include communication circuitry (e.g., wireless communication circuitry) to communicate between components (e.g., joints, etc.) and/or with a remote processor, user smartphone, tablet, etc.

The apparatuses described herein may be operated with a user-control input device, including sensory devices (e.g., EMG, joystick, etc.). For example, the apparatus may include a cuff or band including neuromuscular sensors (e.g., EMG or other equivalent sensor) as control input to control the powered operation of these apparatuses (e.g., artificial, powered limb). The apparatus may be configured to execute pre-programed (e.g., “macro”) movements.

In general, the apparatuses described herein may include one or more sensors for determining the position and/or torque and/or status of one or more joints formed as part of the apparatus (e.g., finger joint, wrist joint, elbow joint, etc.).

For example, described herein are apparatuses including a robotic elbow assembly. These elbow assemblies may be light weight and may have a low power requirement with a high load output. For example, the apparatuses described herein may include an elbow assembly including an antagonistic drive transmission. In some examples the antagonistic drive transmission may be a twisted-fiber antagonistic drive transmission, although other antagonistic drive transmissions may be used (e.g., push-pull antagonistic drive transmissions, etc.).

For example, a motor-driven artificial limb device may include: a limb joint; a first limb movably coupled to the limb joint; a motor; and an antagonistic drive transmission coupling the motor to the first limb and configured to move the first limb relative to a second limb coupled to the limb joint. The antagonistic drive transmission may be a twisted-fiber antagonistic drive transmission.

For example, a motor-driven artificial limb device may include: a limb joint; a first limb distal to the limb joint and configured to move relative to the limb joint; a motor at or proximal to the limb joint; and an antagonistic twisted-fiber drive transmission configured to move the first limb relative to the limb joint, wherein the antagonistic twisted-fiber drive transmission further comprises at least two sets of fibers that are configured to twist in opposite directions during operation of the antagonistic twisted-fiber drive transmission.

In some examples a motor-driven artificial limb device may include: a limb joint; a first limb extending distally from the limb joint; a second limb extending proximally from the limb joint; a motor at or proximal to the limb joint; an antagonistic twisted-fiber drive transmission driven by the motor and configured to move the first limb relative to the second limb, wherein the antagonistic twisted-fiber drive transmission comprises a first set of fibers and a second set of fibers; and a capstan comprising a first cam lobe engaging with the first set of fibers or a first transmission cable length coupled in series with the first set of fibers, and a second cam lobe engaging with the second set of fibers or a second transmission cable length coupled in series with the second set of fibers, wherein the first and second cam lobes are configured to alter the effective radius traveled by the first and second sets of fibers or the first and second transmission cable lengths around the capstan.

Any appropriate motor may be used, including (but not limited to) a geared DC motor, a gearless motor, a brushless motor, etc. The motor may be within the limb joint. In some examples the motor may be within the fulcrum of the limb joint (e.g., the elbow).

In any of these examples the antagonistic twisted-fiber drive transmission may comprise two or more winding bobbins. In some examples the apparatus includes a pair of counter-rotating shafts coupling the motor to the two or more winding bobbins of the antagonistic twisted-fiber drive transmission. In some examples the counter-rotating shafts are not used, and the fibers are pre-wound in opposite directions, which may simplify the gearbox. The antagonistic twisted-fiber drive transmission may comprise a set of fibers arranged to twist around each other, and a second set of fibers arranged to twist around each other, wherein the first set of fibers is configured to twist in a first direction when the second set of fibers is configured to twist in a second direction that is opposite from the first direction. The antagonistic twisted-fiber drive transmission may couple to a capstan. The first set of fibers may couple in series to a first transmission cable length through a first connector, and wherein the first transmission cable length couples to the capstan; further wherein the second set of fibers couples in series to a second transmission cable length through a second connector, and wherein the second transmission cable length couples to the capstan. The capstan may comprise a first cam lobe coupled to the first set of fibers and a second cam lobe coupled to the second set of fibers, wherein the first and second cam lobes are configured to alter the effective radius around the capstan by the first set of fibers or a first transmission cable length coupled in series to the first set of fibers around the capstan and the second set of fibers or a second transmission cable length coupled in series to the second set of fibers around the capstan.

In general, the capstan may include a pair of cam lobes engaged with the at least two sets of fibers or to one or more transmission cables coupled in series to the sets of fibers, wherein the cam lobes are configured to alter the effective radius traveled by the sets of fibers or one or more transmission cables around the capstan. The cam lobes may be particularly useful in distributing and making more uniform the forces and bending movement as the antagonistic drive transmission (e.g., twisted fiber) transmission operated, since the foreshortening and/or expansion of the twisted fibers may be nonlinear. The cam lobes may counteract this nonlinearity.

The first limb may be configured to bend about the limb joint by more than 60 degrees (e.g., 65 degrees or more, by 70 degrees or more, by 75 degrees or more, by 77 degrees or more, by 80 degrees or more by 85 degrees or more, by 90 degrees or more, by between 60-09 degrees, between 65-90 degrees, between 70-90 degrees, etc.).

The first set of fibers and the second set of fibers may comprise an ultra-high-molecular-weight polyethylene.

As mentioned, in some examples the first limb comprises a prosthetic forearm. In some examples the artificial limb comprises a robotic limb. In some examples the artificial limb comprises a prosthetic limb.

Any of these examples may include a tensioner coupled to the antagonistic twisted-fiber drive transmission to remove slack from the antagonistic twisted-fiber drive transmission. The motor may be coaxial with the limb joint.

Although any appropriate tensioner may be used, in some examples the tensioner is a tensioner device for a drive transmission having a chassis, the device comprising: a pulley configured to apply tension to a transmission cable length; a tensioner body rigidly coupled to the pulley; a rachet rigidly coupled to the tensioner body and configured to allow movement of the tensioner body in a first direction relative to the chassis to increase the tension applied to the transmission cable length, but not in a second direction that is opposite the first direction; and a bias coupled to the tensioner body and configured to apply a force between the tensioner body and the chassis resulting in tension of the transmission cable length.

The tensioner body may comprise a plate. The rachet may comprise a plurality of teeth engaging a plurality of teeth or slots formed by or coupled to the chassis. The rachet may be a continuous rachet. The continuous rachet may comprise a plate and a dowel or ball.

Any of these apparatuses may include a second rachet rigidly coupled to the tensioner body and configured to allow movement of the tensioner body in the first direction relative to the chassis, but not in the second direction. The chassis may comprise an arm of an artificial limb. The artificial limb may include a prosthesis or robotic limb.

For example, a tensioner device for a drive transmission having a chassis may include: a first pulley configured to apply tension to a first transmission cable length; a second pulley configured to apply tension to a second transmission cable length; a first tensioner body rigidly coupled to the first pulley; a second tensioner body rigidly coupled to the second pulley; a bias coupled to the first tensioner body and the second tensioner body and configured to apply a force between the first tensioner body and the second tensioner body; and a first rachet rigidly coupled to the first tensioner body and configured to allow movement of the first tensioner body in a first direction to increase the tension applied to the first transmission cable length, but not in a second direction that is opposite the first direction.

Any of these apparatuses may include a second rachet rigidly coupled to the second tensioner body and configured to allow movement of the second tensioner body in the second direction relative to the chassis to increase the tension applied to the second transmission cable length, but not in the first direction. The bias may be coupled to the first tensioner body and the second tensioner body through the chassis. The first tensioner body may comprise a first plate and/or the second tensioner body comprises a second plate. The first rachet may comprise a plurality of teeth engaging a plurality of teeth or slots formed by or coupled to the chassis or to the second tensioner body. The first rachet may be a continuous rachet. For example, the continuous rachet may comprise a plate and a dowel or ball (that may grip a strap, wire, etc. of the rachet at any position, rather than at discrete positions).

Any of these apparatuses may include an additional one or more rachets rigidly coupled to the first tensioner body and configured to allow movement of the first tensioner body in the first direction, but not in the second direction. The chassis may comprise an arm of an artificial limb. The artificial limb may comprise a prosthesis or robotic limb.

Any of these apparatuses may alternatively or additionally include a wrist assembly as described herein. For example, an artificial limb apparatus (e.g., device) may include: a cylindrical wrist mount coupled to a first end of a first limb; a spherical wrist head rotatably held with the cylindrical wrist mount; a first pair of Bowden cables each coupled to the spherical wrist head and configured to rotate the spheroidal wrist head relative to a long axis of the cylindrical wrist mount; a second pair of Bowden cables each coupled to the spherical wrist head along a first meridian of the spherical wrist head and configured to rotate the spheroidal wrist head relative to an x-axis of the cylindrical wrist mount; a third pair of Bowden cables each coupled to the spherical wrist head along a second meridian of the spherical wrist head and configured to rotate the spherical wrist head relative to a y-axis of the cylindrical wrist mount; wherein the x-axis, the y-axis and the long axis are all perpendicular to each other, and wherein the first meridian is radially offset from the second meridian by between 80-100 degrees at an equator of the spherical wrist head; and one or more motors configured to drive the first, second and third pairs of Bowden cable assemblies.

In any of these examples the second pair of Bowden cables may each extend within a channel along the first meridian on opposites sides of the spherical wrist head, and wherein the third pair of Bowden cables each extend within a channel along the second meridian on opposites sides of the spherical wrist head. The cylindrical wrist head may comprise a hollow ball having an opening at a proximal end and a distal end of the hollow ball. Where a hand (e.g., fingers) are coupled to the end of the arm/wrist the mount and/or control components (e.g., wires, power, etc.) may be passed through the openings at the proximal and distal ends without preventing movement of the wrist apparatus.

The apparatus may include an inner housing interposed between the cylindrical wrist mount and the spherical wrist head and configured to rotate with the spherical wrist head. The inner housing may be coupled to the spherical wrist head by the second and third Bowden cable assemblies. The one or more motors may be positioned at or proximal to a second end of the first limb. Any of these apparatuses may include or more cable tensioners for each of the first, second, and third pairs of Bowden cables, wherein the one or more cable tensioners are spring loaded assemblies that are configured to maintain a constant tension on each of the first, second and third pairs of Bowden cables.

The one or more motors may comprise a single motor configured to drive all of the first, second and third pairs of Bowden cables.

Any of these apparatuses may include an outer housing at least partially covering the spherical wrist head and configured to limit movement of the spherical wrist head relative to the cylindrical wrist mount. The shaft may extend distally from the spherical wrist head. Each of the first, second and third pairs of Bowden cables may include an PTFE outer tubing.

Any of these apparatuses may include a feedback sensor configured to sense relative movement between the spherical wrist head and the cylindrical wrist mount. The feedback sensor may comprise a hall effect sensor having a magnetic element within the spherical wrist head.

The cylindrical wrist mount may include a ring coupled to the first end of the first limb.

These apparatuses may include one or more cable sensors configured to detect extension and retraction of an inner cable or inner cables of each of the first, second or third pairs of Bowden cables.

For example, a motor-driven artificial limb device may include: a cylindrical wrist mount coupled to a first end of a first limb; a spherical wrist head rotatably held with the cylindrical wrist mount; a first pair of Bowden cables each coupled to the spherical wrist head and configured to rotate the spheroidal wrist head relative to a long axis of the cylindrical wrist mount; a second pair of Bowden cables each coupled to the spherical wrist head along a first meridian of the spherical wrist head and configured to rotate the spheroidal wrist head relative to an x-axis of the cylindrical wrist mount; a third pair of Bowden cables each coupled to the spherical wrist head along a second meridian of the spherical wrist head and configured to rotate the spherical wrist head relative to a y-axis of the cylindrical wrist mount; wherein the x-axis, the y-axis and the long axis are all perpendicular to each other, and wherein the first meridian is radially offset from the second meridian; a wrist inner housing interposed between the cylindrical wrist mount and the spherical wrist head and configured to rotate with the spherical wrist head; wherein the second and third pairs of Bowden cables pass through channels on the wrist inner housing and couple to the spherical wrist head; and one or more motors configured to drive the first, second and third pairs of Bowden cable assemblies.

The wrist inner housing may be coupled to the spherical wrist head by the second and third pairs of Bowden cables. As mentioned any of these apparatuses may include one or more cable tensioners for each of the first second and third pairs of Bowden cables, wherein the one or more cable tensioners are spring loaded assemblies that are configured to maintain a constant tension on each of the first, second and third pairs of Bowden cables. The one or more motors may comprise a single motor configured to drive all of the first, second and third pairs of Bowden cables. The outer tubing of each Bowden cable of the first, second and third pairs of Bowden cables may comprise a PTFE tubing.

Any of these apparatuses may include a feedback sensor configured to sense relative movement between the spherical wrist head and the cylindrical wrist mount. The feedback sensor may comprise a hall effect sensor having a magnetic element within the spherical wrist head. The apparatus may include one or more cable sensors configured to detect extension and retraction of an inner cable or inner cables of each of the first, second or third pairs of Bowden cables.

Also described herein are powered finger joints. In general, these powered finger joints may be combined in series to form finger and in parallel to form multiple fingers of a hand or other appendage. For example, a powered compact finger joint device may include: a motor; a nested dual-stage cycloidal reduction gear having a first cycloidal stage receiving input from motor, the first cycloidal stage having a first eccentric input shaft, a first cycloidal pinion, and a ring gear housing, wherein the first cycloidal pinion couples to a second cycloidal stage eccentric input shaft, wherein the second cycloidal stage eccentric input shaft couples to a second cycloidal stage pinion driving a floating ring gear within a cycloidal housing; and an output arm coupled to the floating ring gear and configured to move within an output channel through the cycloidal housing; wherein the nested dual-stage cycloidal reduction gear is configured to provide a gear reduction of between about 12:1 to about 25:1.

The motor and the nested dual-stage cycloidal reduction gear may be arranged side-by-side and have approximately the same length and same diameter. The length may be 25 mm or less (e.g., 22 mm or less, 20 mm or less, 19 mm or less, 18 mm or less, 17 mm or less, etc.). T the width may be the same as or less than the length. In some examples the width may be 30 mm or less (27 mm or less, 25 mm or less, 22 mm or less, 20 mm or less, etc.). The thickness (“height”) of the combined motor and nested dual-stage cycloidal reduction gear may be less than either the height or the length.

Any of these apparatuses may include a motor output gear coupling the motor to the first eccentric input shaft of the first cycloidal stage. The output channel may extend laterally around a side of the cycloidal housing.

In any of these apparatuses, unlike traditional cycloidal gears, the first cycloidal stage may be unbalanced. For example, each stage may include just a single pinion.

One or more sensors may be used. For example, the apparatus may include one or more sensor configured to sense a position of the output arm. The apparatus may include a sensor configured to detect torque (e.g., of the output arm and/or another finger joint coupled thereto).

The apparatus may include an output platform pivotally mounted to a lateral side of the device.

Any of these apparatuses may include control circuitry configured to control operation of the motor. The control circuitry may be mounted to a lateral side of the device.

In general, a plurality of powered compact finger joints may be coupled together to form one or more robotic fingers. For example, a robotic finger apparatus may include: a pair of powered compact finger joint devices connected in series, wherein each powered compact finger joint device comprises: a motor; a nested dual-stage cycloidal reduction gear having a first cycloidal stage receiving input from motor, the first cycloidal stage having a first eccentric input shaft, a first cycloidal pinion, and a ring gear housing, wherein the first cycloidal pinion couples to a second cycloidal stage eccentric input shaft, wherein the second cycloidal stage eccentric input shaft couples to a second cycloidal stage pinion driving a floating ring gear within a cycloidal housing; and an output arm coupled to the floating ring gear and configured to move within an output channel through the cycloidal housing, wherein the nested dual-stage cycloidal reduction gear is configured to provide a gear reduction of between about 12:1 to about 25:1; wherein a second of the pair of powered compact finger joint devices is rigidly coupled to an output platform, further wherein the output platform is pivotally coupled to a first of the pair of powered compact finger joint device so that the output arm is drives rotation of the output platform relative to the first powered compact finger joint device.

Any of these apparatuses may include a third (or more) powered compact finger joint device coupled to the second of the pair of powered compact finger joint devices through a second output platform, wherein the second output platform is rigidly coupled to the second powered compact finger joint device, further wherein the second output platform is pivotally coupled to the second powered compact finger joint device so that the output arm of the second powered compact finger joint device drives rotation of the second output platform relative to the second powered compact finger joint device.

Any of the features mentioned above for the powered compact finger joints may be used. For example, the motor and the nested dual-stage cycloidal reduction gear within each powered compact finger joint device may be arranged side-by-side and have approximately the same length and same diameter (e.g., the length may be 20 mm or less). A motor output gear within each powered compact finger joint device may couple the motor to the first eccentric input shaft of the first cycloidal stage. The output channel within each powered compact finger joint device may extend laterally around a side of the cycloidal housing.

These apparatuses may include one or more sensors configured to sense a position of the output arm of each of the powered compact finger joint device. For example, any of these apparatuses may include a sensor configured to detect torque between each powered compact finger joint device. The apparatus may include control circuitry on at least one of the powered compact finger joint device configured to control operation of the motor. The control circuitry may be mounted to a lateral side of the first powered compact finger joint device.

All of the methods and apparatuses described herein, in any combination, are herein contemplated and can be used to achieve the benefits as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the methods and apparatuses described herein will be obtained by reference to the following detailed description that sets forth illustrative embodiments, and the accompanying drawings of which:

FIG. 1A shows one example of an apparatus (e.g., a prosthetic arm apparatus) as described herein, which may include one or more of: a powered elbow joint, a powered wrist joint and/or powered finger joints ad described herein.

FIG. 1B is an example of a prosthetic apparatus as described herein.

FIG. 1C is another example of a prosthetic apparatus as described herein.

FIG. 2A schematically illustrates one example of an elbow joint having an antagonistic drive transmission as described herein.

FIG. 2B illustrates the operation of one example of a twisted-fiber drive.

FIGS. 3A-3C illustrate one example of a twisted fiber antagonistic drive transmission as described herein.

FIG. 4A illustrates one example of a twisted fiver antagonistic drive transmission.

FIGS. 4B-4E illustrate an example of an antagonistic drive transmission within an example limb having a limb joint, in which progressively more features are shown removed to illustrate the antagonistic drive transmission.

FIGS. 5A-5C illustrate the elbow region of an example limb including an antagonistic drive transmission as described herein.

FIGS. 6A-6B show an example of an elbow joint as described herein.

FIG. 7 is a graph illustrating the cam radius vs. joint angle for one example of an antagonistic drive transmission.

FIG. 8 shows an example of a cam surface that may be used with a twisted-fiber antagonistic drive transmission.

FIG. 9A shows another example of a cam (e.g., a deal cam) for a twisted-fiber antagonistic drive transmission as described herein.

FIG. 9B shows an example of a cam having an error-correcting groove or channel extending along the curved length of the cam. The error-correcting groove corrects error from the primary antagonistic cam mechanism (e.g., tangent error) arising from the proximity of a guide pulley that routes the cable to the upper and/or lower cam surfaces. The grove traverses the curved surface of the cam at an angle to the midline of the curved cam surface.

FIG. 10 is a graph showing an example of fiber bundle contraction vs. motor turns for various examples of a twisted-fiber antagonistic drive transmission.

FIG. 11 schematically illustrates a limb including a twisted-fiber antagonistic drive transmission with a self-tensioning apparatus (tensioner).

FIG. 12 schematically illustrates one example of a tensioner as described herein.

FIG. 13 schematically illustrates one example of a tensioner as described herein.

FIG. 14 schematically illustrates an example of a tensioner as described herein.

FIG. 15 schematically illustrates an example of a tensioner as described herein.

FIG. 16 schematically illustrates an example of a tensioner as described herein.

FIG. 17A schematically illustrates an example of a tensioner as described herein.

FIG. 17B is an exploded view of the tensioner shown in FIG. 17A.

FIGS. 18A-18C schematically illustrate an example of a tensioner as described herein.

FIGS. 19A-19F illustrate one example of a powered wrist joint as described herein.

FIG. 19A shows a front perspective view, FIG. 19B shows a back perspective view, FIG. 19C shows a section through the device of FIG. 19A, and FIG. 19D shows an exploded view. FIG. 19E shows an example with the outer cylindrical wrist mount, nosecone and outer housing removed.

FIGS. 20A-20B show another example of a powered wrist joint as described herein.

FIG. 21 is a section through one example of a powered wrist joint.

FIG. 22 is a partially transparent perspective view of a powered wrist joint.

FIG. 23 illustrates an example of a parallel capstan (e.g., driver) for driving movement of a powered wrist joint.

FIGS. 24A-24B illustrate an example of a driver for a powered wrist joint that includes a single motor.

FIGS. 25A-25C illustrate examples of a powered finger formed by a plurality of powered compact finger joints as described herein. FIGS. 25A-25B show a finger with two powered joints, while FIG. 25C shows an example with a single powered joint.

FIG. 26 shows an example of a compact finger joint.

FIG. 27 shows an exploded view of the compact finger joint of FIG. 26A.

FIGS. 28A and 28B show exploded views (front and back, respectively) of the nested dual-stage cycloidal of a powered compact finger joint as described herein.

FIG. 29A shows a section through an example of a compact finger joint similar to that shown in FIG. 26.

FIG. 29B shows an end view of the compact finger joint of FIG. 29A with the cover (removed).

FIG. 30A shows a sectional perspective view of an example of a compact finger joint similar to that shown in FIG. 26.

FIG. 30B shows an enlarged view of a portion of a compact finger joint.

FIG. 31 shows another example of a compact finger joint in perspective view with a portion of the device made transparent.

FIG. 32 shows another example of a compact finger joint in perspective view with a portion of the device made transparent.

FIG. 33 shows an end view of a portion of a compact finger joint showing various sensors that may be included.

FIG. 34 shows an end view of another end of a compact finger joint showing sensors that may be included.

FIG. 35 shows another example of a compact finger joint in perspective view with a portion of the device made transparent.

FIG. 36 shows an end view of an example of a compact finger joint including an output platform for coupling the apparatus to a second finger joint.

FIG. 37 illustrates a compact finger joint in perspective view showing the control circuitry (e.g., control PCB) and connection to an adjacent compact finger joint.

FIG. 38 shows an example of a plurality of compact finger joints coupled together to form a finger as described herein.

### DETAILED DESCRIPTION

Artificial limbs as described herein may refer to prosthetics and/or robotic arms. For example, the methods and apparatuses described herein may be used as part of a powered prosthetic apparatus to be worn by a user or they may equivalently be a part of a robotic apparatus that may be operated remotely and/or automatically. The apparatuses (devices and/or system, including artificial limbs) described herein may include one or more of any of: elbow joint assemblies (e.g., robotic elbow joint assemblies), wrist assemblies (e.g., robotic wrist assemblies) and hand/finger assemblies (including robotic finger assemblies).

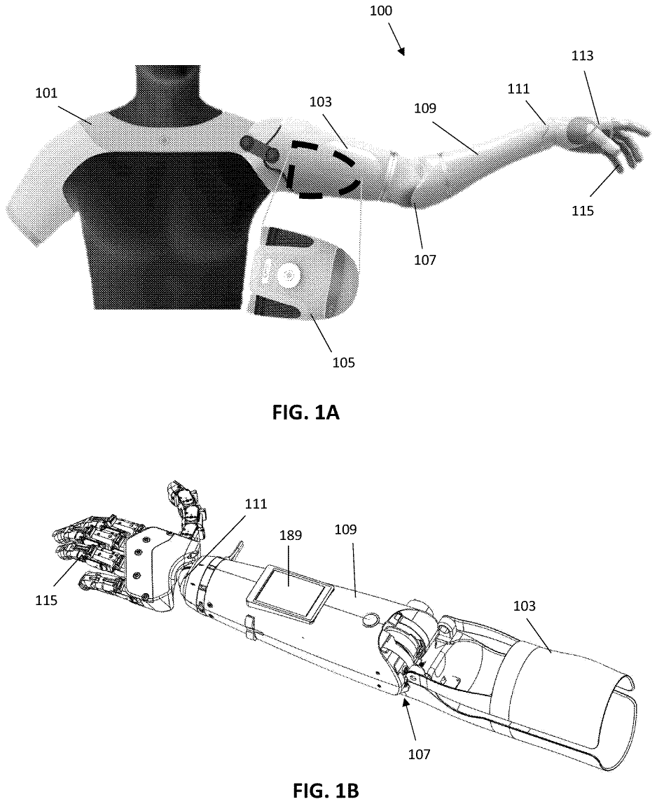

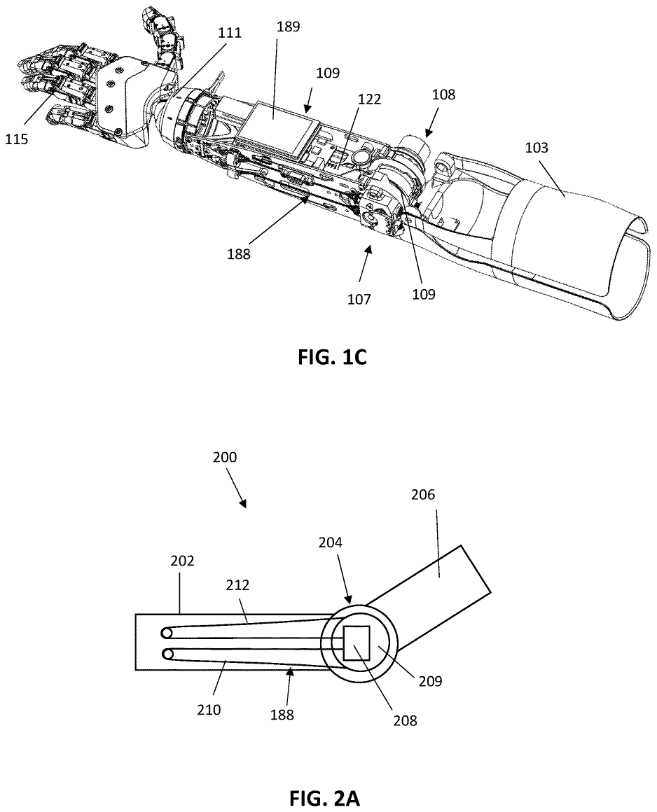

For example, FIG. 1A illustrates an example of an apparatus configured as a prosthetic device to be worn by a user. The user may be, for example, an amputee. In this example the apparatus may include a harness or yoke 101 to which the limb apparatus 100 may be coupled. The user may also wear a neuromuscular interface, in this example configured as a cuff 105 which may be applied over the skin in order to detect input (e.g., signals, including electromyographic, EMG, signals) to control the apparatus. The limb apparatus 100 may include an upper arm portion 103 that may be coupled to the user's torso and/or the yoke 101 and may include an elbow joint 107 linked to bot the upper arm portion 103 and a forearm portion 109. In any of these examples the elbow joint 107 may be powered, as described herein. The forearm 109 may include an outer housing enclosing the powered and unpowered components, and couples to a wrist joint 111, which may be a powered wrist joint, as described herein. The wrist joint may connect to a hand with fingers 115, which may be powered, and a palm.

Any of these apparatuses may include one or more processors for controlling operation of these apparatuses. The one or more processors (e.g., configured as a controller or controller) may be integrated into the apparatus, e.g., within the upper arm and/or forearm and/or wrist and/or hand and/or finger(s). These apparatuses may generally include a power supply (rechargeable power supply, wall power adapter, etc.), and may include communication circuitry (e.g., wireless communication circuitry) to communicate between components (e.g., joints, etc.) and/or with a remote processor, user smartphone, tablet, etc. A variety of different sensors may be included. In general, the powered components (elbow, wrist, fingers, etc.) may include sensors to detect forces, including torque, acting on the powered component, and/or may include sensors for detecting position (absolute position and/or relative position). Sensors may include force sensors, accelerometers, etc., and in some case proximity sensors (including optical, ultrasound, electrical (e.g., ultrawideband, UWB, sensors, etc.), etc., may be integrated into the apparatus, including on figures and/or palm 113 and/or back of the hand, side(s) of the hand, wrist, forearm, etc.

As mentioned, the apparatuses described herein may be operated with a user-control input device, including sensory devices (e.g., EMG, joystick, etc.). For example, the apparatus may include a cuff or band including neuromuscular sensors (e.g., EMG or other equivalent sensor) as control input to control the powered operation of these apparatuses (e.g., artificial, powered limb). The apparatus may be configured to execute pre-programed (e.g., “macro”) movements.

In general, the apparatuses described herein may include one or more sensors for determining the position and/or torque and/or status of one or more joints formed as part of the apparatus (e.g., finger joint, wrist joint, elbow joint, etc.).

For example, FIG. 1B shows an example of an apparatus configured as a prosthetic device, including powered fingers 115 coupled to a powered wrist 111 that may controllably move relative to a forearm 109 region that may itself be moved relative to an upper arm 103 region coupled to the user's body. In this example the apparatus may receive user's control input through a neural interface, such as a cuff 105, as mentioned above (e.g., EMG input) and/or may receive input wirelessly (e.g., via a user's phone or other device), by receiving input from the user's other arm, and/or from remote input. In FIG. 1B the apparatus also includes a touchscreen/display 189 that may act as both an output (outputting information, and/or control setting) and/or may also receive input from the user.

The apparatuses described herein are configured to be particularly lightweight and may have a low power requirement with a high load output. For example, as shown in FIG. 1C, in which the protective housing over the forearm in FIG. 1B has been removed, the apparatus includes a powered elbow assembly configured as an antagonistic drive transmission 188. In some examples, as shown in FIG. 1C, the antagonistic drive transmission may be a twisted-fiber antagonistic drive transmission, although other antagonistic drive transmissions may be used (e.g., push-pull antagonistic drive transmissions, etc.). The powered joints (e.g., fingers, wrist, elbow) may be powered by one or more batteries 122 that may be include in either or both the upper arm region 103 and/or the forearm 109 region. In general, it may be beneficial to position the batteries (e.g., rechargeable batteries, regenerative batteries, etc.) closer to the elbow joint region and/or in the upper arm region 103 so that the weight is closer to the user's center of mass. In some examples the batteries (or some of the batteries) may be part of the yoke or harness holding the apparatus on the user's body. Batteries may be integrated and/or may be swappable (e.g., as a battery pack), for immediate use. In some cases, an on-board battery may be included in addition to a swappable battery back, and the on-board battery may be recharged by the pack.

Thus the motor-driven artificial limb apparatuses (e.g., devices, systems, etc.) may include: a limb joint (e.g., a powered elbow joint 107); a first limb (e.g., a forearm 109) movably coupled to the limb joint; a motor 108 (which may be within the elbow joint); and an antagonistic drive transmission 188 coupling the motor 108 to the first limb and configured to move the first limb relative 109 to a second limb (e.g., upper arm region 103) coupled to the limb joint. The antagonistic drive transmission may be a twisted-fiber antagonistic drive transmission.

### Elbow Transmission

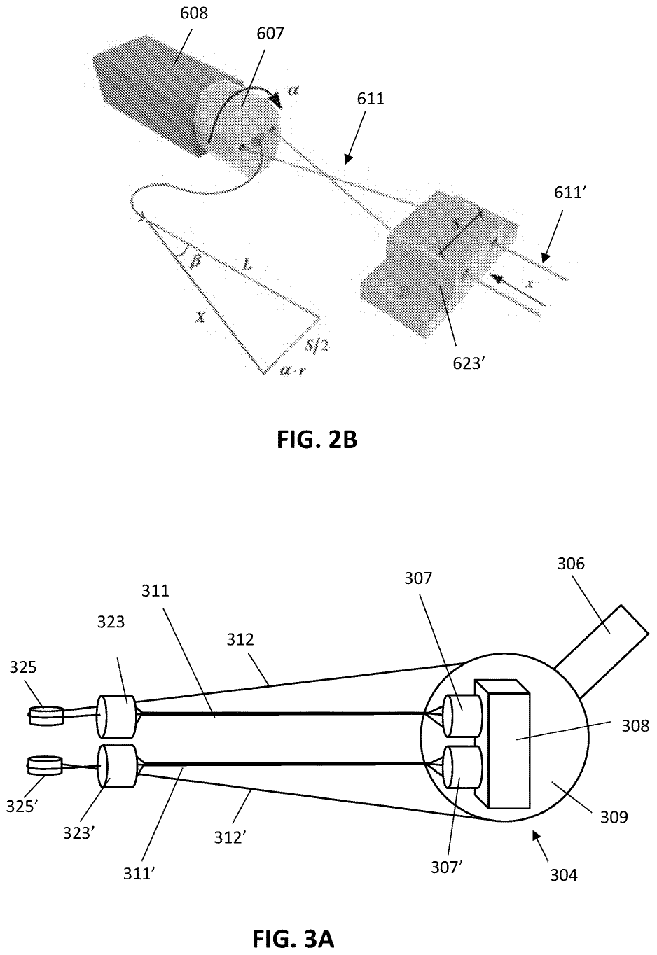

As mentioned, any of the apparatuses described herein may include an antagonistic drive transmission, such as a twisted fiber drive and control feedback mechanism. FIG. 2A schematically illustrates a generic antagonistic drive transmission 200 that includes a motor 208 (in this example, positioned within elbow or limb joint 204), a cam 209 that may rotate when driven by the antagonistic fiber drive 188, which also includes fibers including a first fiber or set of fibers 212 and a second, or counter, fiber or set of fibers 210. The first and second fibers (or sets of fibers) are driven in opposite directions by the motor (or in some examples multiple motors, though a single motor may be preferred), and may be re-directed around one or more pulley(s) and/or capstan(s) and may couple to the cam. In Any of these examples the fiber may be a bundle of fibers and/or a belt, strand, etc. The first 212 and second 210 fibers may be separate lengths of the same fiber (e.g., wrapped around the cam) or they may be different fibers (separately attached to the cam). The antagonist fiber drive may move the first limb 202 relative to the second limb 206 (e.g., move the forearm relative to the upper arm). The motor may be rigidly secured to the first limb, the elbow joint and/or the second limb. In some examples the motor is fixed relative to a frame within the first limb around which the fibers or lengths of fibers (e.g., the first fiber/length of fiber 212 and the second fiber or length of fiber 210) extend. The fibers/lengths of fiber may wind around the cam and antagonistically pull to bend/extend the first limb 202 relative to the second limb 206. In general, the first fiber/length of fiber may be pulled (e.g., towards the motor in FIG. 2A) to “contract,” while the second fiber/length of fiber may relax or release and “extend” to move the forearm in a first rotational direction about the elbow joint or this movement may be reversed by pulling the second fiber/length of fiber (e.g., towards the motor in FIG. 2) and extending the first fiber/length of fiber. This operation allows the first fiber/set of fiber to operate antagonistically.

For example, the antagonistic fiber drive may be a twisted fiber drive that is used to move the first limb relative to the second limb (e.g., move the forearm relative to the upper arm) around an elbow joint, achieving a surprisingly large force while requiring only a minimal volume and weight. A twisted fiber transmission may transfer power through the elbow joint to the forearm. In some examples a motor (e.g., a geared DC motor) is located inside the elbow joint coaxial with rotation, as shown in FIG. 1C. This gear motor may drive two fiber winding bobbins. In some examples the gear motor may optionally couple to the bobbins through a pair of counter rotating shafts (or alternatively, directly). Twisted fiber transmissions may offer a very high mechanical advantage (e.g., effective transmission ratios) and high bandwidth with silent and forceful operation. They are also non-linear in that the fiber bundle contraction is not linearly related to the number of rotations. There is less contraction per turn at the lower number of turns than when the bundle is wound up. For example, the third turn may provide 1 mm of displacement and the 20^{th} turn provides 3 mm. For example, FIG. 2B illustrates a schematic showing the use of a winding bobbin and a “separator” block, also referred to herein as a collimator or collimator block since it may keep the fibers straight coming out of the wind zone. In FIG. 2B, the set of fibers 611 coming out of the separator or collimator (block 623′) may pull as the fiber is wound by the motor 608 driving rotation of the bobbin 607. Winding the set of fibers 611 causes them to foreshorten, pulling the separated fibers 611′ towards the separator (e.g., the collimator 623′), as shown.

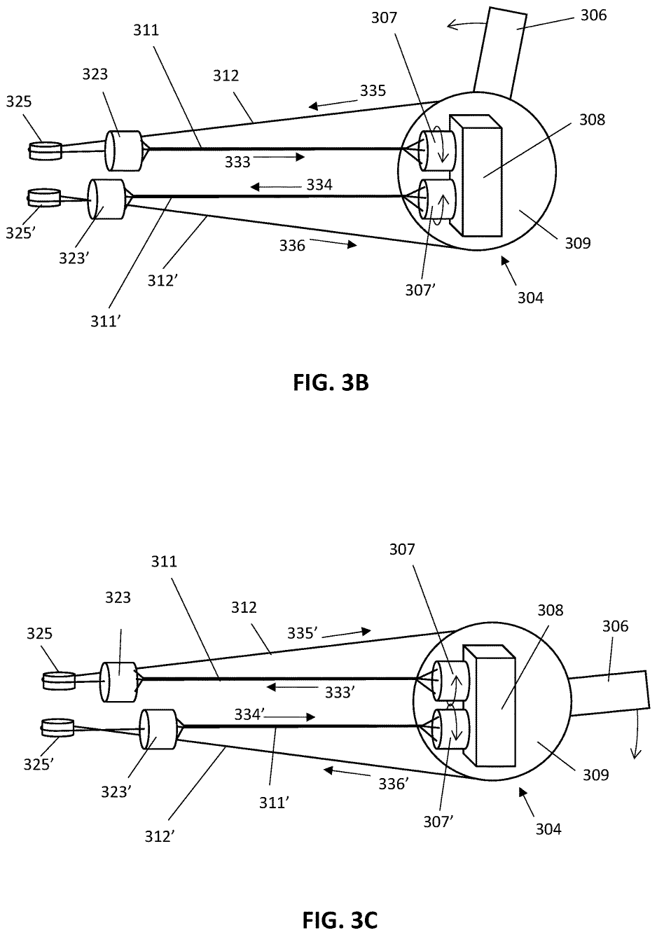

FIGS. 3A-3C illustrate one example of a twisted fiber transmission as described herein. In FIG. 3A, the antagonist fiber drive is configured as twisted fiber transmission including a motor 308, shown proximal or in the elbow region (limb joint 304). The same elbow joint includes a rotating cam 309 that is rigidly coupled with a second limb 306 (e.g., upper arm region). The cam may rotate relative to the first limb (e.g., forearm region) in which the fibers of the twisted fiber transmission extend (not shown in this schematic). The motor 308 may drive a pair of bobbins 307, 307′ in opposite directions (alternatively in some examples multiple motors may be used, include separate motors that may be coordinated by a controller). The bobbins may each couple to a plurality of fibers 311, 311′ that extend from the rotating bobbins to a separator 323, 323′ (also referred to herein as a collimator). The in some examples the same fibers forming the first set of fiber 311 may extend from the separator/collimator and may be re-directed (e.g., via one or more pulley(s) 325, 325′, capstans, etc.) back to the cam to extend partially or completely around the cam. The fiber/length of fiber forming the first and second transmission cables 312, 312′ may be formed of the same fibers as the first and/or second sets of fibers, or they may be formed of a separate length(s) of material. The antagonistic first set of fibers 311 and second set of fibers 311′ may be wound in opposite directions in operation, so that winding the first set of fibers in a first direction unwinds the second set of fibers. The effect of winding and unwinding may shorten (when winding up) or lengthen (when unwinding) the sets of fibers; the resulting antagonistic forces may be applied to the cam via a cam surface that translates the winding up/unwinding of the opposing sets of fibers and the resulting linear motion and into rotational movement. The shape of the cam may be configured as described in detail herein in order to even out the nonlinear contraction/release as the fibers are wound and unwound.

FIGS. 3B-3C illustrate operation of an example of a twisted fiber drive system as part of a powered elbow joint. In FIG. 3B, the motor 308 drive rotation (e.g., clockwise rotation) of the first bobbin 307 to wind up the first set of fibers 311, causing them to foreshorten, as shown by arrow 333. Concurrently, the second bobbin 307′ is driven in the opposite direction (e.g., counterclockwise rotation), causing the second set of fibers 311′ to unwind, lengthening, as shown by arrow 334. The resulting antagonistic movements of the first and second sets of fibers results in a linear movement of the first transmission cable 312 in a first direction 335 and the linear movement of the second transmission cable 312′ in a second direction 336. This linear movement is translated into a rotational movement of the elbow joint capstan or pulley 309, which also includes the cam surface(s) 309′. The transmission cables/lengths of cable may be fixed to the capstan/cam surface 309/309′; as one length of transmission cable moves in the first direction, the second length of transmission cable moves in the second direction and the capstan/pulley is rotated to move the elbow joint and therefore the limb. The linear movements of the transmission cables will be different as the motor rotates, but this difference may be accounted for by the different cam surfaces on the elbow joint; the elbow joint pulley to which the cam or cams is coupled forming the cam surfaces on which the first and second transmission cables travel. FIG. 3C shows the reverse movement, in which the first bobbin 307 is rotated in the second direction to unwind, and the second bobbin 307′ is rotated in the first direction to wind up, reversing the linear movement 333′ of the first set of fibers and the linear movement 335′ of the first transmission cable 312 and the linear movement 334′ of the second set of fibers and the linear movement 336′ of the second transmission cable 312′.

In the example shown in FIGS. 3B-3C the separator/connector may move linearly (in/out relative to the long axis of the limb) but is prevented from rotating. For example, the separator/connector 323, 323′ may be held in a track (not shown). The transmission cable (e.g., wire, belt, fiber, etc.) may be formed of the same fibers forming the sets of fibers 311, 311′ or may be formed of a separate fiber or lengths of fiber that are coupled to the separator/connector. Alternatively in some examples the separator/connector may act as a fixed (relative to the frame of the first limb, not shown), and the same fibers of the first set of fibers may form the first transmission cable or may be coupled (after the separator/connector 323) to a separate transmission cable or length of cable.

One way to use a twisted fiber transmission is to have the fiber tension do work against a load like gravity, e.g., the weight of the forearm, plus the load being lifted. As long as there is enough load, the fiber can be unwound by the control software/firmware/hardware. However, it may be better to operate against an additional spring force. This way, the fiber always has a restoring load to unwind as long as the firmware commands the motor to turn in the unwinding direction. If the arm is being operated in a vertical orientation where gravity and the load are not offering restoring force as the fiber is unwound, the spring may provide the necessary force. However, such an arrangement with a load spring may require the motor to work against the spring in order to do work to lift a load, thus diminishing overall efficiency and adding the weight of a spring.

Alternatively, as described in FIGS. 3A-3C, two light-weight fiber bundles may be antagonistically opposed where one winds while the other unwinds. In this case, no energy is stored, or work done on any Hookean spring elements. Any arrangement of initial wind conditions may result in binding as one fiber takes up slack faster than the other produces it. If each bundle starts off with exactly one half the number of maximum turns (to reach a desired stroke) then in the center it will work symmetrically, but as the stroke reaches +/−some value, it may gradually bind up and the load on the motor and tension in the bundles may reach a stall condition.

As described herein, a packaging efficient method of terminating fiber bundles in a way to rotate the elbow joint +/−77 degrees or more may configure the fiber drive so that the fibers wrap around both sides of the elbow capstan (e.g., of approximately 40 mm outside diameter) over a cam surface that can account for this discrepancy. In some examples both fiber bundles may be crimped to more easily managed flexible steel wire rope cables with plastic out coating. These cables may be terminated at the center of the capstan. One may pull while the other is pulled, and visa-versa.

Thus, the challenge of binding of the fibers may be solved in the apparatuses herein by altering the instantaneous radius of contact with the fibers. The capstan surface may be formed into two cam lobes. These lobes may exactly alter the effective radius at a given angle such that the extra slack in one cable is balanced by the other. One side of the capstan may have a radius R and the other side a conjugate radius R′ that is its compliment. The cams may be symmetric. The cam profiles may be derived from actual fiber twist versus change in length behavior and the resulting dR/dtheta may be identified.

For example, in order to lift approximately 20 pounds in the palm, the fiber tension may be about 1000N or 250 pounds. In some examples the fiber strength (e.g., of a Dyneema® fiber) has a factor of safety from a stress standpoint that is sufficient. The rigidity of the arm structure and the ability to maintain tension in the fibers may be maintained by using one or more tensioners, as described herein.

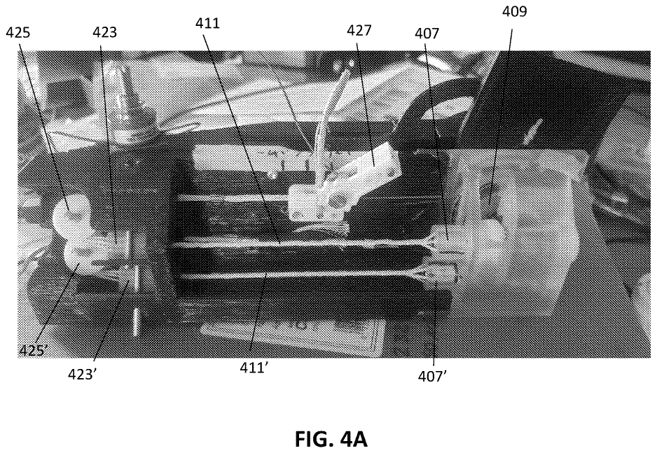

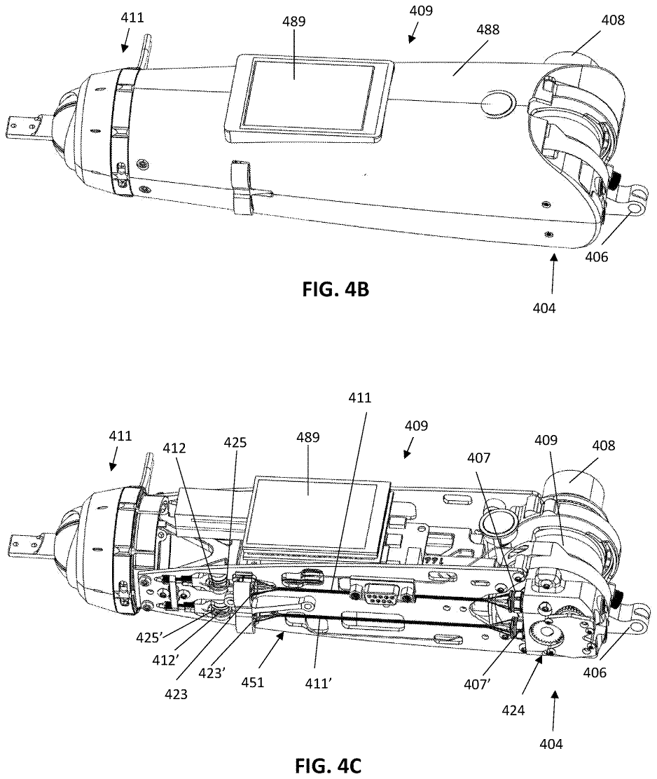

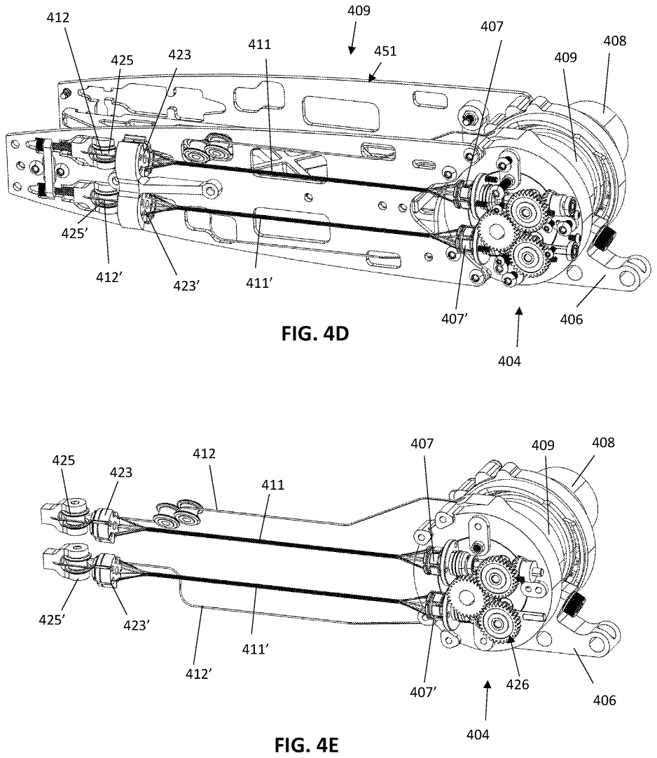

The antagonistic fiber drive configuration described above was tested with a linear PID control system and performed very well in terms of smoothness, overshoot, and stability. A simple potentiometer was used to measure joint angle and a second potentiometer was used as a reference target command input. FIG. 4A illustrates an example prototype of a twisted fiber drive including a motor driving a first and second bobbin 407, 407′, twisting/untwisting a first and second sets of fibers 411, 411′ each set feeding into a separator/connector 423, 423′ (e.g. collimators) that are fixed to the frame and collimate the strands to form the transmission cables that extend over pulleys 425, 425′ and back to a capstan with dual cam surfaces 409. A tensioner 427 is also shown to maintain tension on the transmission cables. The example shown in FIG. 4 was used to validate the cam profiles, the tensioner, and general viability of the transmission concept. It was constructed of 3D printed SLA and FDM parts, and accordingly, was only able to operate at low loading conditions.

FIGS. 4B-4E illustrate an example of a twisted fiber drive similar to that shown in FIG. 4A within a robotic limb. The twisted-fiber drive may be integrated into frame 451 of the limb to drive movement of the forearm 409 relative to the limb joint 404 and an upper limb (not shown). FIG. 4B shows the assembled robotic arm including the forearm 409, cover/housing 488 and a limb joint 404 including an attached second limb connector 406. The second limb connector may couple to the upper arm portion. The robotic limb may include a display 409 as mentioned, for input/output (including status outputs, power, etc.) and/or control. A powered wrist joint 411 may also be included and may couple to powered or unpowered hand/finger members. A motor 408 may be part of the limb joint and may drive the twisted fiber transmission as shown in FIG. 4C. In FIG. 4C the housing cover 488 is removed from the apparatus of FIG. 4B, showing the frame to which the gear box 424 including gears to transfer rotary movement by the motor 408 into rotation of the first and second bobbin 407, 407′. The bobbins wind and unwind a first and second sets of fibers 411, 411′ that are, in turn collimated by the separator/couplers 423, 423′ so that the fibers may extend as transmission cables 412, 412′ over one or more pulleys 425, 425′ and couple to the capstan with cam surfaces 409 to drive movement of the forearm relative to the second limb connector 406.

FIGS. 4D and 4E show the same apparatus with components not directly part of the drive system (twisted fiber drive system) removed, to better show the interconnection of the components of the twisted fiber drive. In FIG. 4D the wrist and display as well as internal control circuits and gear box cover 424 have been removed, leaving the arm frame 451 exposed. In FIG. 4E the arm frame has been removed.

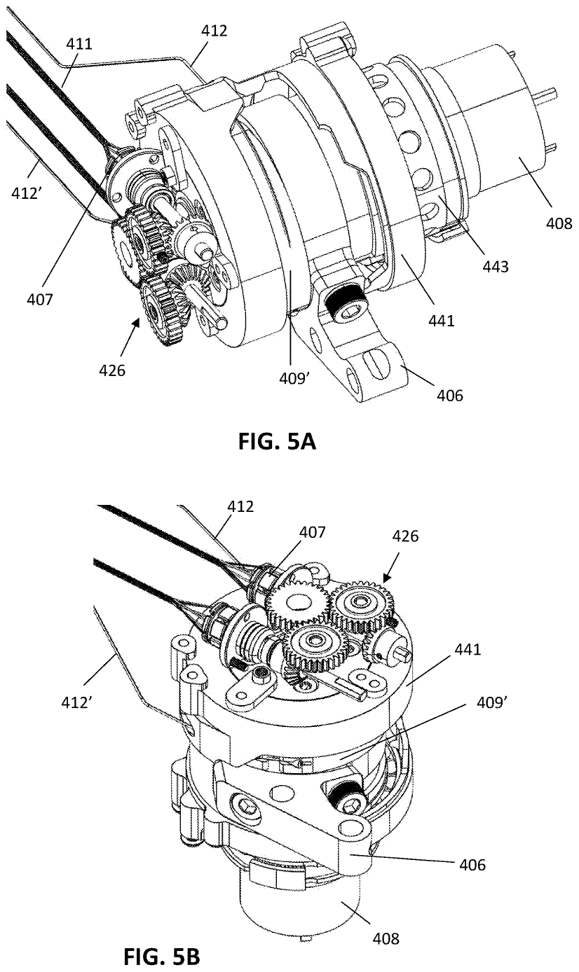

FIGS. 5A, 5B and 5C show enlarged views of the elbow joint region of FIG. 4E, showing the arrangement of the transmission cables 412, 412′, bobbin 407, cam 409′ on the capstan and elbow output tube 443 and elbow housing 441. The motor transmission gears 426 may be coupled to the elbow housing 441. FIG. 5C shows the connection between the motor including a driving gear 633 and the gears 426 driving he bobbins.

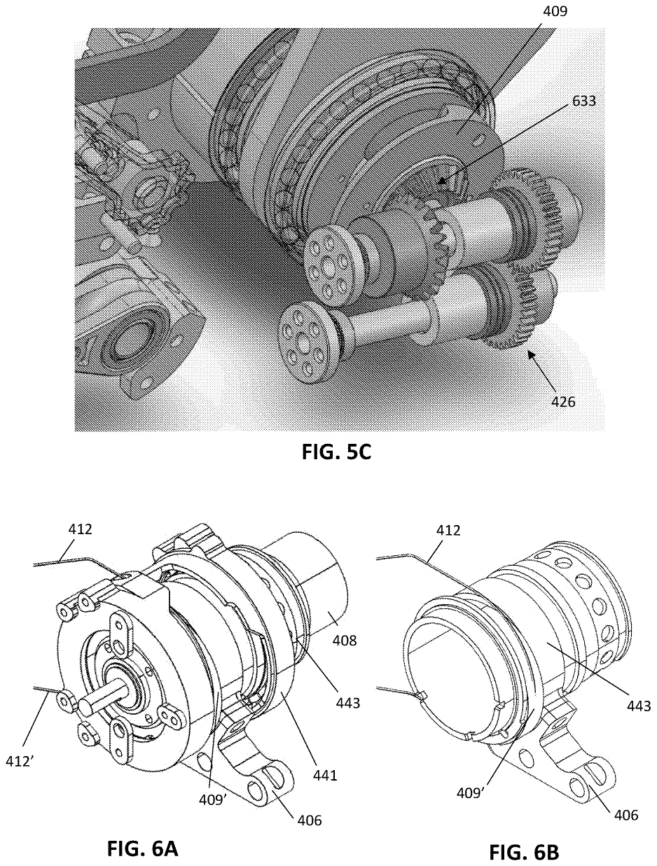

FIGS. 6A-6B illustrate the elbow joint. In FIG. 6A the joint is shown with the elbow housing 441, and motor 408 positioned within the elbow output tube that may form or may be coupled to the capstan and cam 409 (including cam surfaces 409′). In FIG. 6B the motor 408 and elbow housing have been removed.

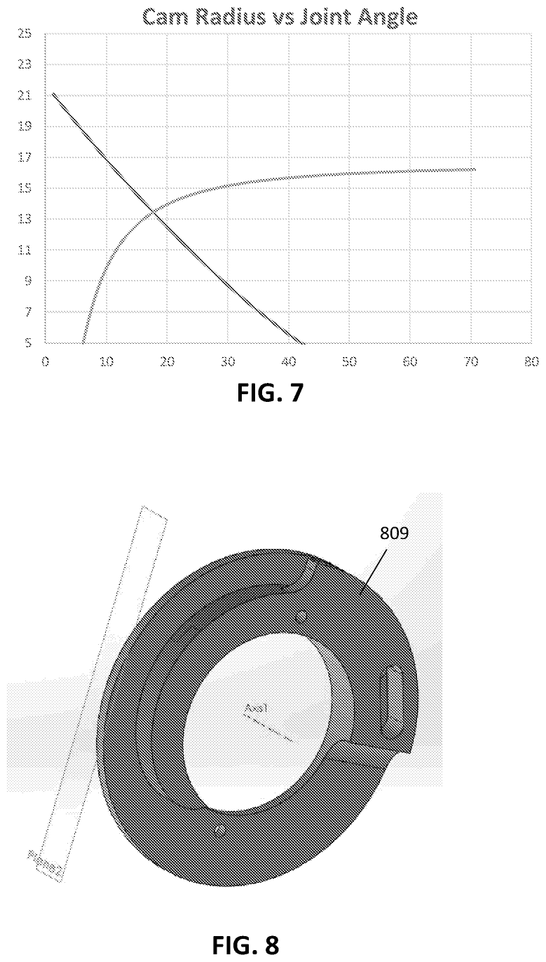

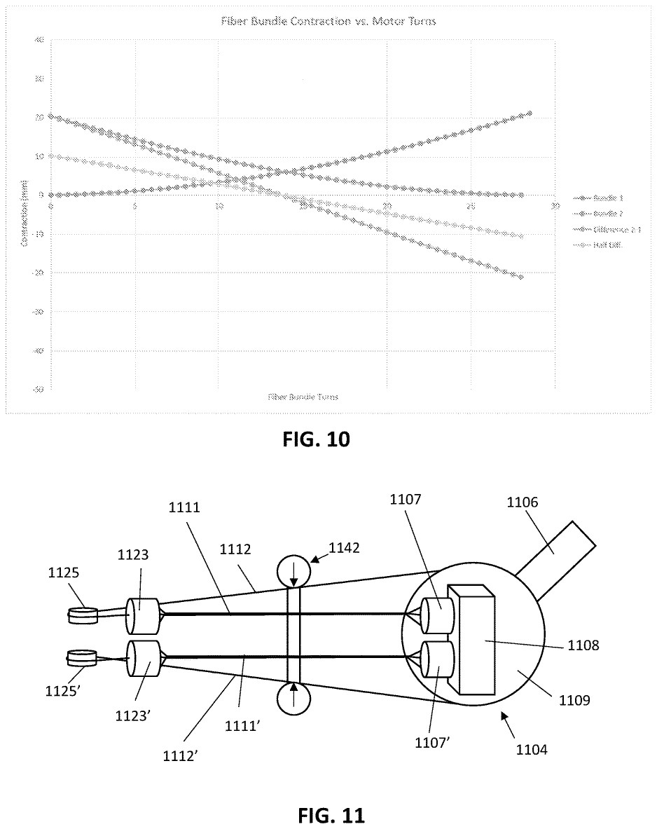

FIGS. 10, 7, 8 and 9A-9B illustrate examples of capstans and cam surfaces that may be used. As described above and illustrated in FIG. 7, the joint angle may be made more regular by selecting the cam surface (e.g., cam radius) on the outside of the capstan so that as the twisted fibers wind up and unwind, resulting in uneven linear travel of the fibers, the cam surfaces (radiuses) may adjust to correct for this, as shown. FIG. 10 illustrates the effect of fiber bundle contraction vs motor turns, showing the differences that may be accounted for by the capstan and cam surfaces.

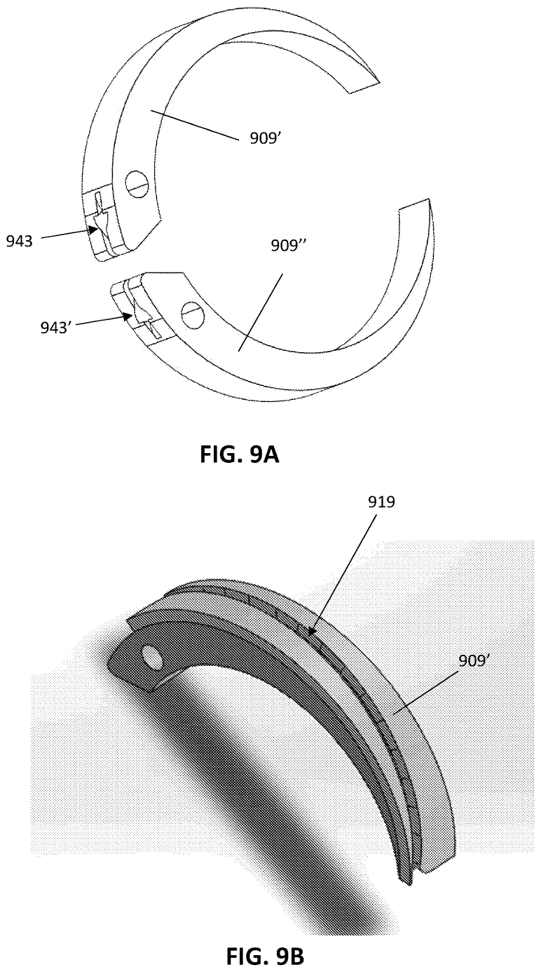

FIG. 7 shows cam radius vs. joint angle for one example of a cam, such as the capstan and cam surfaces shown in FIG. 8. FIG. 8 illustrates an example of a capstan with integrated cam surfaces 809. FIGS. 9A and 9B show examples of just cam surfaces 909′, 909″ that may be used. These example cam surfaces may be coupled to the cylindrical capstan. Alternatively, they may be integrated into a capstan or pulley, as shown in FIG. 8. In FIG. 9A the cam surfaces may also include coupling/attachment regions 943, 943′ for coupling to the filaments.

In FIG. 9B, the cam surface includes a channel or curved groove that is configured as an error-correcting curved groove. The error-correcting curved groove 919 may be configured to correct an error in the primary antagonistic cam mechanism. This error, referred to as the tangent error, may arise from the proximity of a guide pulley that routes the transmission cable to upper and lower cams (see, e.g., FIG. 4E). In some examples, these two pulleys may be far away and the error caused may be smaller, however if they are closer, the error may be significant. However, an error-correcting curved groove 919 or channel having an axial arrangement transverse to the cam surface may correct this error. For example, in FIG. 9B the tangent error-correcting curved groove extends across the curved cam surface from an upper edge region diagonally along the curved cam surface to a lower edge region, as shown. This configuration has been found to effectively limit the error.

Any of the apparatuses described herein may include a tensioner. Fiber tension may fall into two categories: first, the initial fiber/cable assembly and setup and second, the maintenance of tension in operation. Any slack in the system is wasted actuator rotation and worse, dead band or backlash in effect that the control system can't tolerate or compensate for smoothly.

Tensioner designs are described in greater detail below. In some examples the tensioner includes a needle bearing clutch and a spring and may include a floating design that is in series with the cable/fiber bundle. One such tensioner can provide slack removal and tension for the whole system of opposing cable/bundles. An alternative design may be configured in series with the cable to be tensioned and may be based on a simple spring and the winding of the cable around a pivot cylinder/capstan. This design also has the added feature of a flange that may be temporarily grounded to the forearm structure to aide in assembly and initial tensioning. This tensioner may be screwed to the forearm frame and crimped cable assembled in place. A turnbuckle on the opposing cable assembly enables initial slack to be completely removed. Then, the tensioning arm is rotated stretching the cable and tensioner spring. When the desired tension is achieved, a locking pin is inserted, and the screws removed. The tensioner now float in series with the cable and the spring maintains tension. The design geometry and spring size may be modified to improve performance.

The tensioner may be configured to rachet any slack out of the system. Slack may result as cables stretch. Virtually all cable materials, including stainless steel, polymeric, e.g., Dyneema® cables, exhibit some stretch over time. Thus, the tensioner described herein may include a combination of ratcheting and a very high spring preload that may be achieved over a long spring extension (e.g., low K value with high preload value). Alternatively or additionally, in some examples the apparatuses described herein may include just a spring (without a rachet) such as a small displacement and high-force spring, (e.g., a Bellevue washer) in combination with a designed “dead-band” in a tensioner that would not rachet within.

FIG. 11 illustrates one example of twisted fiber drive similar to that shown in FIG. 3A but including a tensioner 1142 to apply tension to the first transmission cable 1112 and the second transmission cable 1112′. As in FIG. 3A, the twisted fiber drive includes a motor 1108 driving a pair of bobbins 1107, 1107′ twisting/untwisting the antagonistic first 1111 and second 1111′ sets of fibers that are columnated by a separator/connector 1123, 1123′ into the first 1112 and second 1112′ transmission cables extending over one or more pulleys 1125, 1125′ before coupling to a capstan with cam surfaces 1109 in the limb joint 1104. Driving the twisting/untwisting of the fibers therefore changes the angle between the forearm to which the drive is coupled and the second limb 1106.

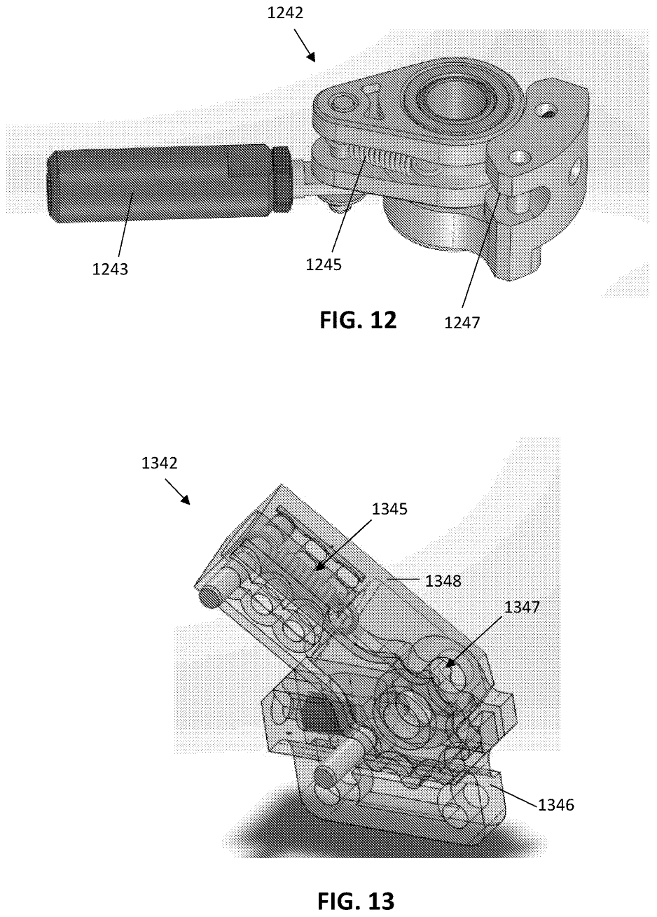

Any appropriate tensioner may be used, including a manual ratcheting tensioner 1242 as shown in FIG. 12, which includes a handle 1243, a bias 1245 and a rachet region 1247. The tensioner 1342 in FIG. 13 also includes a rachet 1347 and bias 1345 arranged in series so that the plate/frame 1348 of the device may be coupled to the chassis/plate frame 1346 through the bias and the rachet.

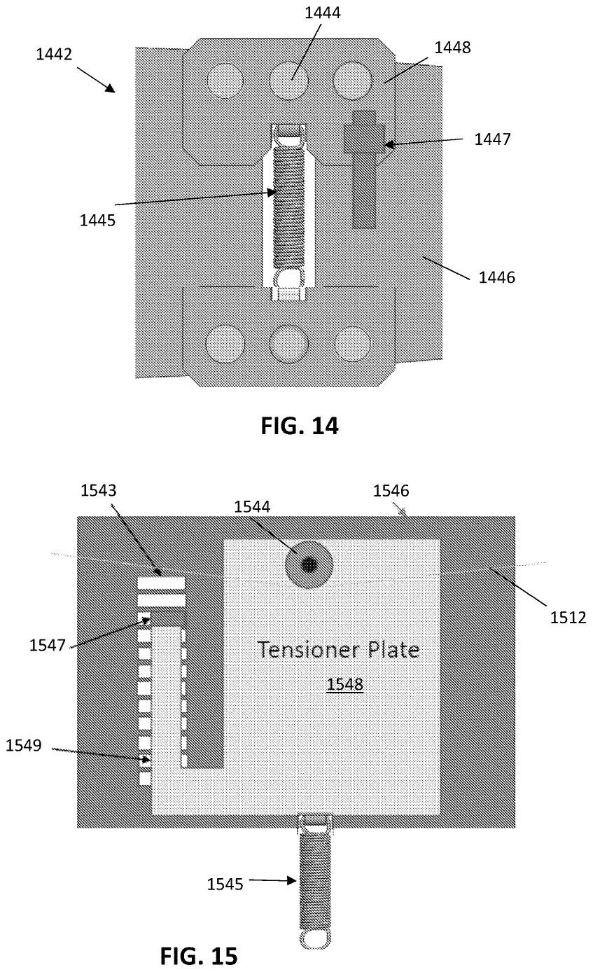

FIGS. 14, 15 and 16 all illustrate different examples of tensioners that may be used. In FIG. 14, the tensioner 1442 may apply tension to a transmission cable (not shown) passing over a pulley 1444; the pulley 1444 is coupled to a tensioner plate/frame 1448 to which both a bias (spring 1445) and rachet 1447. The other end of the rachet connects to the chassis (e.g., plate/frame 1446) of the limb. In FIG. 45 the pulley 1544 may apply tension to the transmission cable 1512 by pulling down via the action of the bias (spring 1545). In parallel a rachet 1547 formed as a discrete rachet from holes in the chassis plate/frame 1546 that engage with teeth on an arm beam 1549. This configuration may allow for relatively low bias force (e.g., springs) to maintain tension, even if the orientation of the tensioner changes relative to gravity, as is likely for a movable prosthetic or robotic arm.

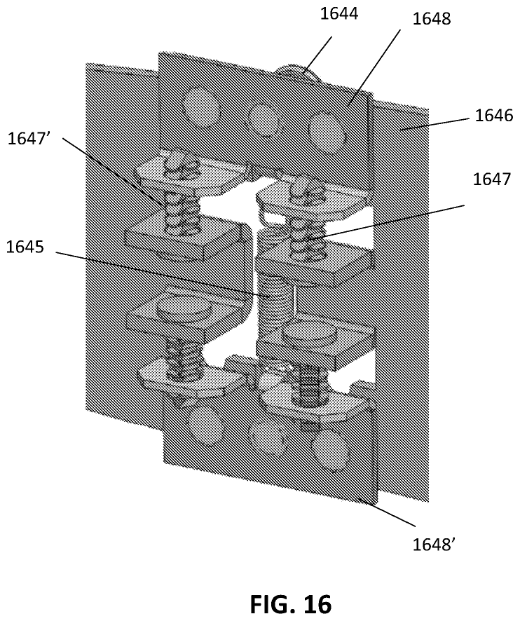

FIG. 16 shows another variation of a tensioner including two sets of rachets 1647, 1647′ that operate in parallel with a bias 1645 to maintain tension on a filament passing over the pulley 1644 coupled to a tension plate 1648, 1648′ relative to a chassis plate/frame 1646.

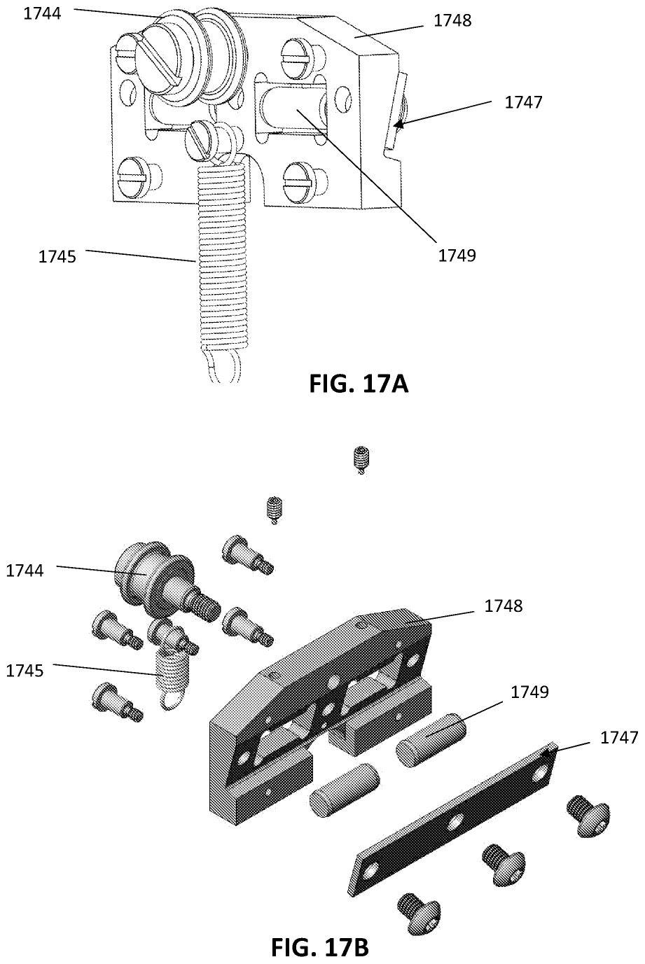

FIGS. 17A-17B show another example of a tensioner including a pulley 1744 coupled to a tension plate 1748 with a bias 1745 and a rachet; the rachet is configured as a continuous rachet with a pair of rachet dowels 1749 and a rachet plate 1747 that secure a filament, belt or other rachet material therebetween. FIG. 17B shows an exploded view of the tensioner of FIG. 17A.

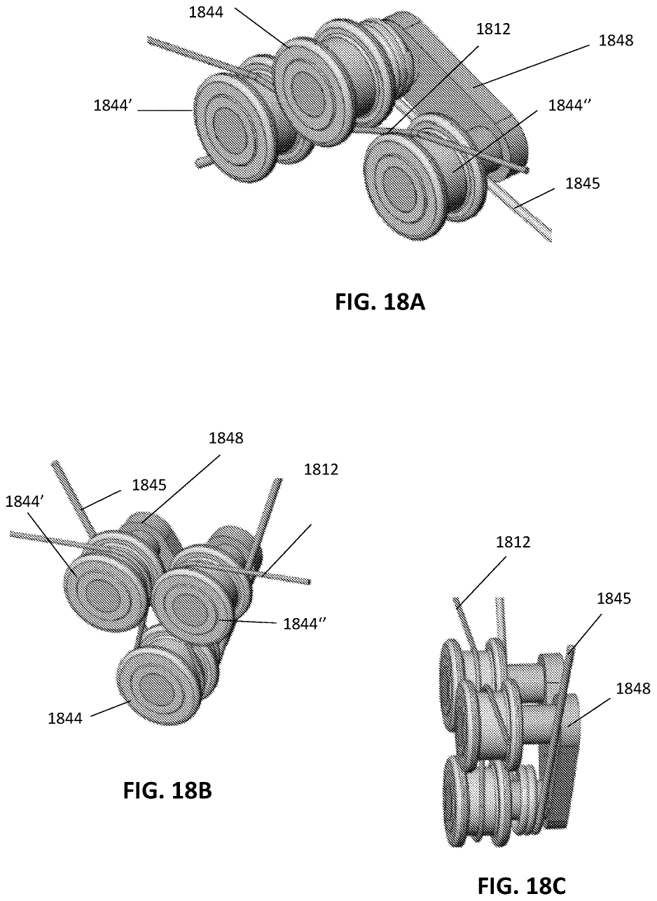

In some examples, multiple pulleys and one or more bias may be used to remove slack from the twisted fiber drives as described herein. In FIGS. 18A-18C the apparatus includes three pulleys 1844, 1844′, 1844″ that may maintain tension of a transmission cable 1812; in this example the bias 1845 may apply force to pull the outer pulleys closer together while driving the inner pulley away (e.g., down) to increase the path length of the transmission cable.

### Wrist Transmission

As mentioned, any of these apparatuses may include a powered wrist joint. The wrist joint may be a ball joint. The wrist joint may be driven to move in two planes (x, y) and may be configured to allow rotation of the joint in the z axis perpendicular to these planes; other movements may be constrained. In addition, the movement of the joint may be constrained. In general, a powered wrist joint may include a cylindrical (e.g., ring-shaped) wrist mount that can be mounted or coupled to a first end of a limb, and a spherical (e.g., ball-shaped) wrist head that is rotatably held with the cylindrical wrist mount. The spherical wrist head may generally be referred to as a ball and may be ball-shaped, but does not have to be a complete sphere; in some examples the distal end region may be open and the proximal end region may be opened, to allow passage through the wrist head and/or attachment of a member, such as a shaft to which another limb and/or hand or other actuator may be attached.

The wrist joint may include an actuator (e.g., cable, gears, etc.) or a pair of antagonistic actuators configured to rotate the spherical wrist head in the long axis extending through the midline of the cylindrical wrist mount. In some examples the actuator for rotating the wrist head relative to the long axis (e.g., z axis) of the wrist mount may be one or a pair of Bowden cables each coupled to the spherical wrist head and configured to rotate the spheroidal wrist head relative to a long axis of the cylindrical wrist mount. In some examples the actuator for rotating the spherical wrist head may be a gear assembly.

The powered wrist joint may also include antagonistic pairs of cables (e.g., Bowden cables) to rotate the spherical wrist head in the x and y plane. For example, it may be particularly beneficial to include a pair of Bowden cables each coupled to the spherical wrist head along a first meridian of the spherical wrist head and configured to rotate the spheroidal wrist head relative to a plane in the x-axis (e.g., the zx plane, or polar angle) of the cylindrical wrist mount and a pair of Bowden cables each coupled to the spherical wrist head along a second meridian of the spherical wrist head and configured to rotate the spherical wrist head relative to a plane in the y-axis (e.g., the zy plane, e.g., azimuth angle) of the cylindrical wrist mount. The x-axis, the y-axis and the long axis (z-axis) are all perpendicular to each other, and the first meridian may be radially offset from the second meridian by between 80-100 degrees at an equator of the spherical wrist head. The wrist joint may also include one or more motors configured to drive the actuator and each of the pairs of cables (e.g., Bowden cable assemblies).

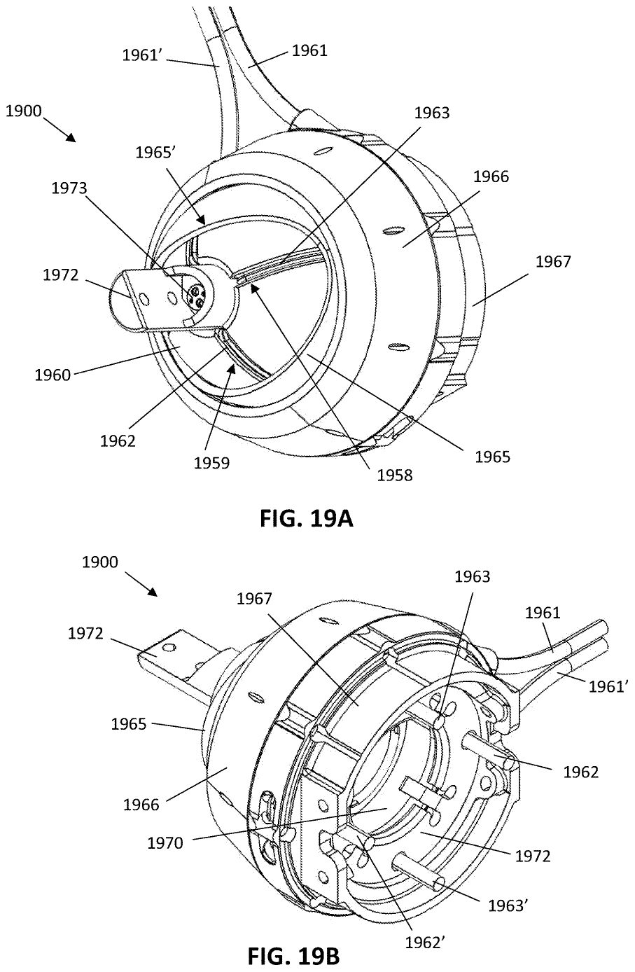

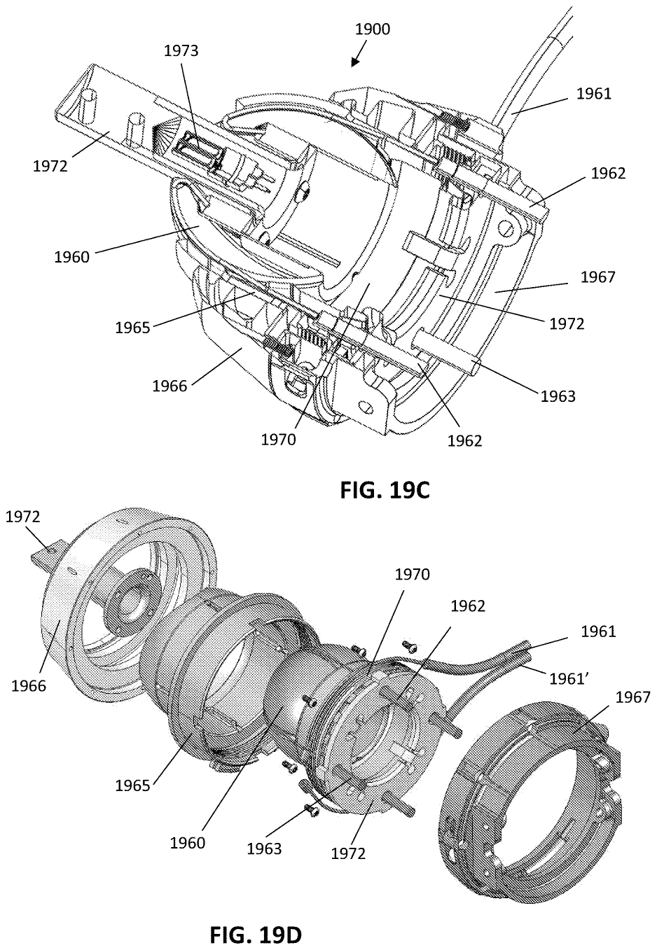

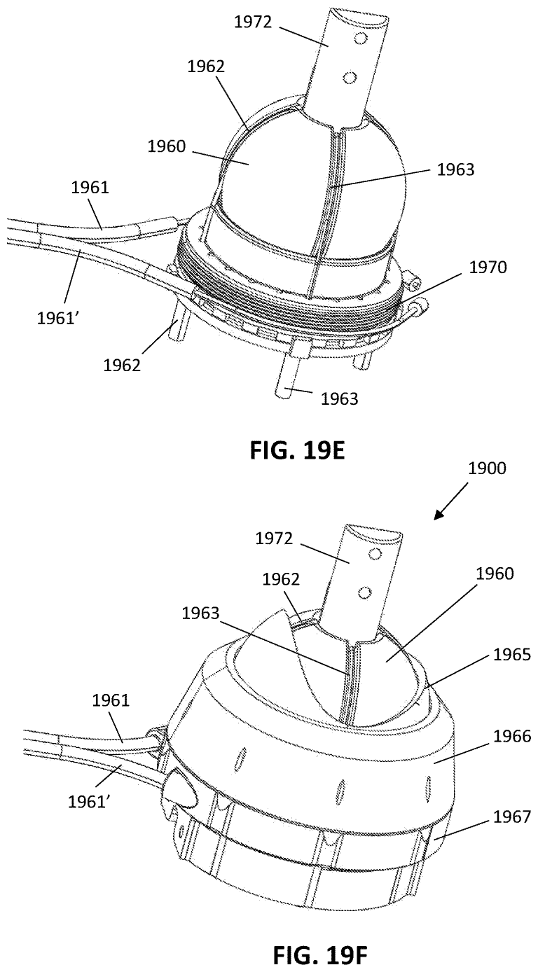

For example, FIGS. 19A-19F shows one example of a powered wrist joint (wrist assembly 1900) including a cylindrical wrist mount 1967, a spherical wrist head 1960 rotatably held within the wrist mount by a wrist inner housing 1970 and a wrist outer housing 1965, and outer wrist nosecone 1966. The wrist head 1960 includes a first meridian 1963 and a second meridian 1959 that intersect at the distal and proximal ends and are separated from each other by 90 degrees at the equator of the wrist head (ball). A shaft 1972 extend from the distal opening through the spherical wrist head in this example and may be configured to include a connector 1973 that may electrical and/or mechanical contact or connection with an additional limb or actuator, such as a hand and/or fingers (e.g., powered fingers) as shown in FIGS. 1B and 1C. The first and second meridians may form channels on or in the outer surface of the spherical wrist head, and the pairs of antagonistic cables (e.g., Bowden cables) may be coupled to the spherical wrist head at these meridians. For example, a first wrist cable 1963 may be Bowden cable that is held at the first meridian 1958 (e.g., within a channel and/or attach to the distal end region of the spherical wrist head. A second, antagonist, wrist cable 1963′ may be a Bowden cable that is held at or in the first meridian 1958′ on the opposite side of the spherical wrist head 1960. The first pair of Bowden cables at the first meridian 1958 may be configured to move the wrist joint in yaw (e.g., changing the polar angle θ (theta) (e.g., the angle with respect to long axis of the wrist mount, z), therefore rotating the wrist head (and the shaft 1972 extending therefrom) in the zx plane. Similarly, the second set of wrist cables 1962 that may be positioned in/at the second meridian 1959 on either side of the spherical wrist head may be actuated to rotate the wrist head (and the attached shaft 1972) in the zy plane, or pitch, changing the azimuthal angle φ (phi) (e.g., angle of rotation from the z plane).

In FIGS. 19A-19F the actuator for rotating the spherical wrist had about the z axis is shown as a pair of Bowden cables (wrist cables 1961, 1961′) that are coupled to the wrist inner housing 1970 to rotate the assembly.

FIGS. 19A and 19B show perspective views of the wrist assembly, while FIG. 19C shows a cross-sectional view and FIG. 19D shows an exploded view, showing one example of the arrangement of the components. The wrist assembly may also include motion-limiting structures such as the wrist outer housing 1965 that includes a jig or cut-out region 1965′ (best seen in FIG. 19A) that prevents rotation of the wrist joint to angles within the cut-out region. The shape of the cut-out region may be modified to allow more or less rotation by changing the shape. The nosecone may also limit the rotation of the spherical wrist head and therefore the shaft extending from the spherical wrist head. The maximum rotation in pitch/yaw (e.g., phi/theta, zx, /zy, etc.) may be between +/−90 degrees, +/−85 degrees, +/−80 degrees, +/−75 degrees, +/−70 degrees, etc.

The wrist assemblies described herein may be lubricated and/or may be formed of a lubricious material (e.g., Teflon, etc.).

In the examples shown, the use of the actuator and/or Bowden cables to control movement of the wrist head 1960 may be particularly advantageous, as it may allow powering and controlling the movement from a more proximal location, such as at or near the elbow joint (or upper arm region) to move wrist using twisted fiber via Bowden cables (and control feedback). This design may therefore allow one or more (e.g., three) motors/actuator, which may be referred to herein a drivers, or when driving Bowden cables, capstan drivers, to control the powered wrist assembly. The one or more motors/drivers may be positioned at the proximal end (or proximal to the proximal end) of the forearm, closer to the elbow and may reduce overall weight, as well as advantageously distribute the weight.

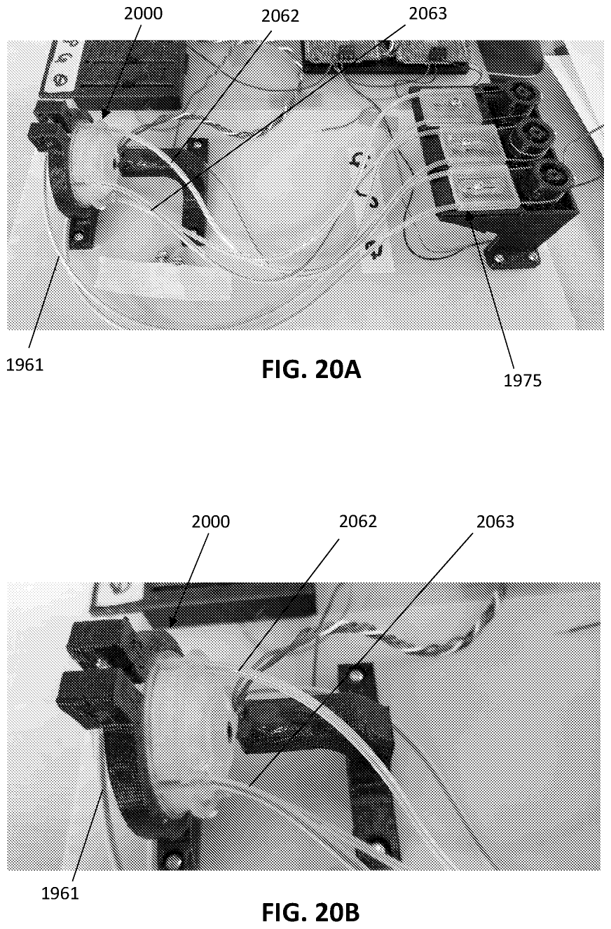

FIGS. 20A and 20B illustrate another example of a wrist assembly 2000 (e.g., a powered wrist joint) including a spherical wrist head that is driven in rotation by three pairs of Boden cables: a first wrist cable pair 1961 driving rotation of the spherical wrist head about the z axis, a second wrist cable pair 2062 driving rotation of the spherical wrist head in pitch (e.g., in the za plane) and a third wrist cable pair 2063 driving rotation of the spherical wrist head in yaw (e.g., in the zy plane). FIG. 20B illustrates an enlarged view of the wrist joint portion of the wrist assembly shown in FIG. 20A. In FIG. 20A the assembly 2000 also includes a capstan/drive portion 1975 driving actuation of the rotation by controlling the Bowden cables.

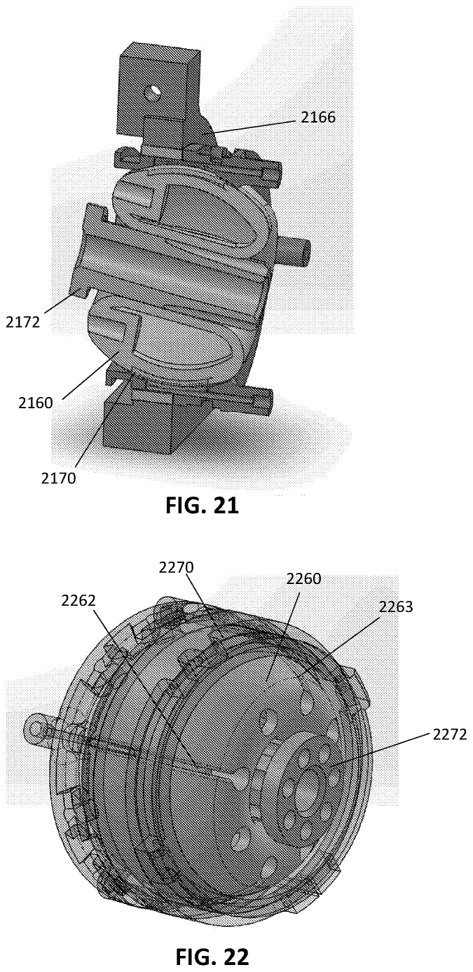

FIG. 21 illustrates a cross-sectional view through an example of a wrist assembly including a spherical wrist head 2160 (e.g., ball) that is driven by the actuators as described above and secured in place so that it may rotate relative to a wrist inner housing 2170 and a nosecone 2166 (e.g., wrist nosecone). A central shaft 2172 passes though the spherical wrist head 2160 and forms a passage through the nosecone, allowing connection to other appendages or components distal to the wrist joint.

FIG. 22 shows a partially transparent perspective view of the wrist assembly shown in FIG. 21. In this example, the spherical wrist head 2260 is rotatably held within an inner housing 2270 and a shaft 2272 extends at least partially through the spherical wrist head 260. The spherical wrist head also includes a pair of meridians forming grooves extending up the length of the spherical wrist head and holding a Bowden cable 2262, 2263 in each. These Bowden cables may be configured to drive rotation of the spherical wrist head.

Bowden cables may have very high stiffness and low friction and are generally capable of both pushing and pulling. When paired on either side of a rotatable armature (e.g., the spherical wrist head), they can provide precise and high bandwidth power transmission taking up very little volume and adding very little weight. Thus, in some examples, a powered wrist joint assembly may include a spherical moving element (spherical wrist head), a cylindrical housing part (cylindrical wrist mount) fixed to the forearm, a rotatable spherical support (e.g., inner wrist housing), and three sets of Bowden cable assemblies with capstans and motors. The mechanism may selectively locate the spherical axis in three degrees of freedom. The powered wrist joint assembly may have a spherical coordinate system arranged for firmware control and/or calculation efficiency. The wrist can be positioned in three principal axes: phi (the rotation angle around the longitudinal axis of the wrist, the axis of the forearm link), alpha x (the rotation about the horizontal reference frame), and alpha Y (the rotation about the Y axis of the reference frame).

In examples including three sets of Bowden cables, the cable tension may be maintained with three separate spring loaded assemblies that ensure near constant tension on each cable. The wrist joint assemblies described herein may be configured so that the control cables that themselves serve as the gimbal guidance for the two rotating degrees of freedom.

That is, the ball (wrist head) may only rotate in X and Y along the path defined by the exit of the steel Bowden cable that is exactly positioned coincidentally with the equator of the moving ball. The four cables (two opposing pairs) may be guided by grooves in the outer ring (first and second meridians) as described above.

In some examples firmware may handle the transformations from a spherical command vector in the spherical reference frame to actual joint rotation angles alpha x and alpha y. The capstan diameters and the drive motor torque and transmission ratio (if a gearbox is used) may provide a mechanical advantage. In some examples the smallest and lightest means to provide a given rotation angle of a capstan that is less than 180 degrees (other examples may include more than 180 degrees, e.g., by wrapping the wire fully around the capstan). The inner diameter of the wrist head (e.g., within the grooves if used) may also determine rotation torque.

In any of the powered wrist assemblies described herein cables may be terminated inside the ball with crimps. Bowden cables may be constructed of a thick walled polymeric (e.g., Teflon PTFE) tubing, such as a tubing having a nominal 3 mm O.D. and 1 mm I.D, and a stainless steel flexible cable, such as a cable with a 0.85-0.95 mm outer nylon jacket. These numbers can vary, but the radial play between the tubing and cable outer diameter may result in slack and should be minimized as described herein.

Any of these apparatuses may include one or more (e.g., two) position control feedback sensor techniques, including sensors. For example, a three-dimensional hall magnetic sensor with on board processing functionality may be positioned axially at the center of the forearm axis in a fixed position. A permanent magnet may be located in the proximal end of the moving spherical ball joint. This sensor may output three vectors that define the position of that magnet relative to the grounded sensor. The apparatus (e.g., firmware) may provide a transformation to separate rot X and rot Y from rot Z. Alternately, each capstan may include an absolute rotation sensor, either a potentiometer or a magnetic rotational hall sensor. Any play in the cable system may be read as a position error. However, not closing the position loop on the actual end effector position but rather relying on a somewhat indirect measurement via cables may be performed instead. This scheme may be used as a backup in the event that the 3D mag sensor gets too complex or if there are any singularities we don't wish to deal with. We could have both measurement systems and use the capstan angles to arbitrate any ambiguities in the 3D output transformation.

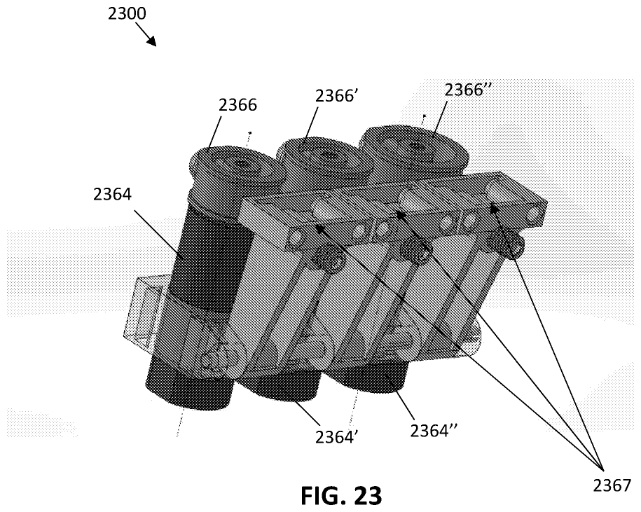

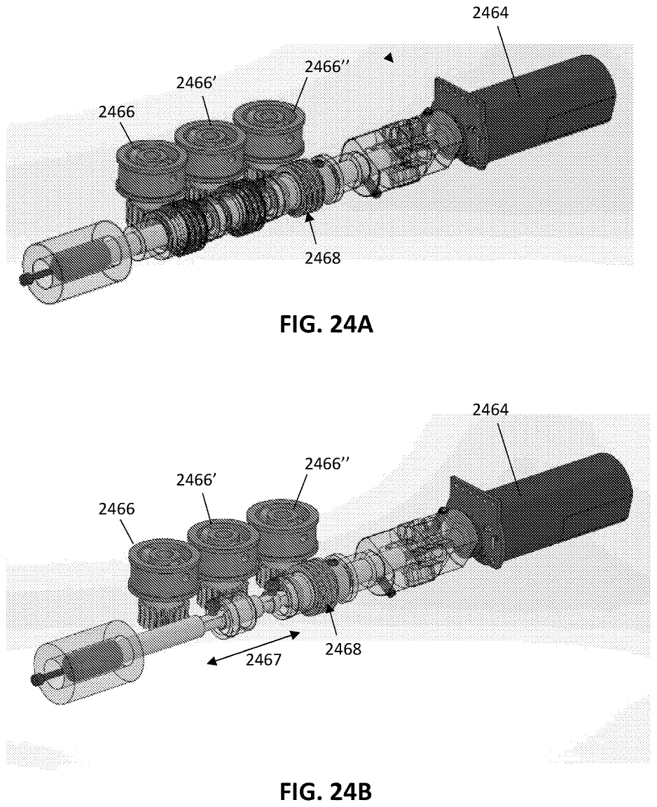

FIG. 23 illustrates a first example of a driver for controlling actuation of a powered wrist joint as described herein. In this example, the driver is configured as a parallel capstan/driver 2300 that includes three motors 2364, 2364′, 2364″ each connected to a capstan 2366, 2366′, 2366″. The group of three wrist servo motors are shown arranged relative to a Bowden cable tensioner 2367 adjacent to each capstan. Alternatively in some examples, a single motor may be used to actuate all three degrees of freedom. For example, FIGS. 24A-24B illustrate an example with a single motor 2464 driving rotation of an engagement gear 2468 that may move axially 2467 to individually drive each of the capstans 2466, 2466′, 2466″ coupled to the Bowden cable sets to move the wrist joint. Thus, a single motor may be axially positioned on a power shaft with a second electromagnetic actuator to selectively position the power shaft in contact with one of three outputs.

Any appropriate motor may be used, such as (but not limited to) a DC geared motor. The apparatus may also include a coupler that transmits torque but allows some axial movement, a compression spring, a rotation encoder to keep track of rotation angle, three sets of worm gears, and a power shaft with mating set screw dogs selectively engaged by a solenoid. In some examples, the spring loaded power shaft may have a neutral position where the center motor is engaged. The solenoid may be, for example, a permanent magnet core and current in one direction through the coil results in pulling the power shaft to the third motor engagement position while reversing the current results in pushing the power shaft to the first motor drive engagement position. In use, this example may use sequential scheduling of the three rotations, but this may be done quickly enough so that it does not significantly impact the user experience and appears to be nearly simultaneous.

### Finger Transmission

Any of the apparatuses described herein may include one or more small (e.g., “finger”) powered joints that may be coupled together sequentially to form fingers. For example, a prosthetic apparatus as described herein may generally include powered or activated fingers (phalanges) having one or more finger joints between finger shaft regions. The finger joints may be powered so that the finger shaft regions may be driven for movement by a motor and a gearing subassembly. These motor and gear subassemblies may be configured specifically to provide a relatively high torque, and therefore output force, while being compact and responsive.

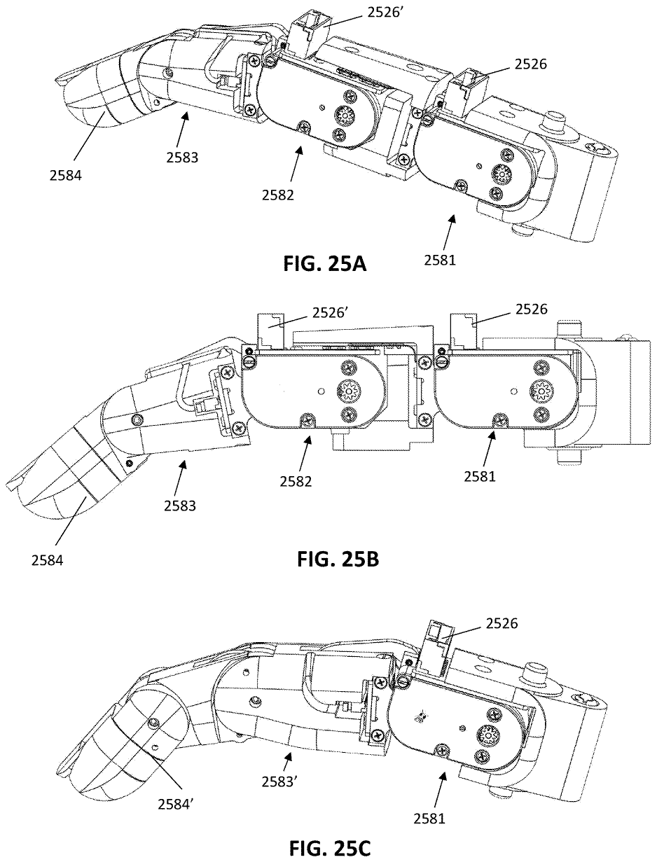

For example, FIGS. 25A-25C illustrate examples of powered fingers including multiple powered joints arranged in series. In FIGS. 25A and 25B a powered finger device includes two powered joints 2581, 2582 as well as an unpowered third joint 2583 and a fingertip region 2584. Each powered joint includes a motor and a reduction gear so that, when controlled by a control input (e.g., via electrical connection 2526, 2526′) the joint may bend relative to an adjacent section of the finger (e.g., up to 110 degrees or more). The passive joints may be configured to bend/unbend as the adjacent powered joint bends (e.g., curling the finger) and unbends. FIG. 25C shows an example of a powered finger that includes a single powered joint 2581 and a passive joint 2583′ and passive fingertip 2584′ region.

To achieve sufficient torque and power in a powered joint that is appropriate for use as a finger joint it is desirable to provide a relatively high gear reduction. However, achieving high levels of gear reduction in a small, compact scale (e.g., less than 20 mm to a side) is difficult. Gear drive units often include a planetary gear reduction. A single-stage planetary gear set is usually only suitable for reductions as large as 1:5 and may require larger sizes. To achieve larger reductions, multiple stages of planetary gear sets are used, but this typically increases the size of the unit and increases the component count and manufacturing costs. Alternatively, described herein are systems (subsystems) including gear drive units that instead use cycloidal gear reduction, which is able to achieve larger reduction ratios than a planetary gear set.

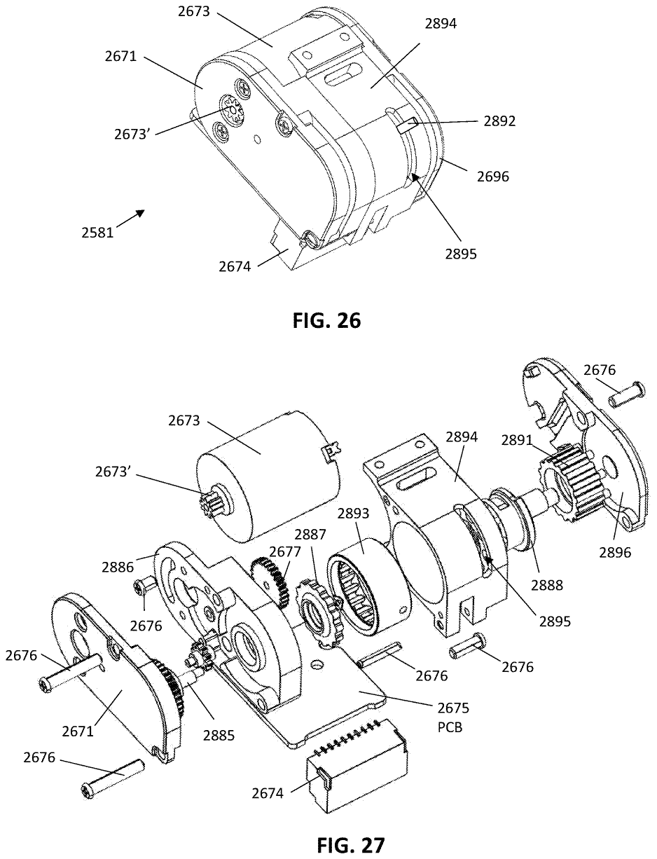

The dual cycloidal gear reduction apparatuses described herein may create a large gear reduction in a small space that fits within the space of a typical finger joint. While typical planetary gear reductions would feature 4-6 stages of 3:1 or 5:1 reductions in 25 mm of length or more, the apparatuses described herein may instead fit two reductions ranging from about 12:1 25:1. For example, a 15:1 1st stage reduction and 17:1 second stage reduction are shown in the examples of FIGS. 26-38. The cycloidal gears forming the compact (powered) finger joints described herein are typically nested, and the second of the two cycloidal gears may be inverted compared to the first cycloidal gear. The first cycloidal gear may receive input from the motor (via a standard gearing connection between the motor output and the input shaft driving the first cycloidal gear). The input to the first cycloidal gear drive a single, unbalanced cycloidal pinon within cycloidal ring gear that is fixed relative to the housing of the cycloidal gear. The output of the first cycloidal gear is a shaft that ten driving input into the second, cycloidal gear nested relative to the first. The second (‘inverted’) cycloidal gear also includes a cycloidal pinion that is eccentrically driven to rotate around a ring gear, however in these examples the second cycloidal ring gear is not fixed but is allowed to float or rotate within the cycloidal gear housing, and the output is coupled to the floating ring gear. For example, the output of the powered finger joint may be a pin or lever that passes through the cycloidal housing, and in particular may pass through a wall of the housing that is on a side (rather than an end) of the powered finger joint (e.g., so that the output extends perpendicular to the long axis of the motor and the nested cycloidal gears. The gear reduction portion may therefore be a nested, dual-stage cycloidal gear reduction which may be positioned adjacent to the motor and may have approximately the same length of a shorter length than the motor (e.g., <20 mm).

Furthermore, although typical cycloidal gears may have pairs of pinions arranged but offset by 180 degrees about the primary drive axis, which may help to dynamically balance the cycloidal gear. In some examples of the apparatuses described herein, however, only a single gear (pinion) may be used, in order to minimize size and part count and to provide better stability to the gear through the use of a pair of bearings.

FIG. 26 shows a perspective view of one example of a powered finger joint including a nested dual-stage cycloidal. The compact powered finger joint 2581 includes a motor 2673 that is coupled side-by-side with a cycloidal housing 2834. As mentioned, an output (shown as a pin 2892 in this example) extends laterally out of the cycloidal housing and travels in a plane transverse to the long axis of the motor, within a channel 2895 (e.g., an output pin channel). One end of the powered finger joint is configured as an end cover 2696 and the oppose end is a cover of the transmission gear box 2671. A motor output 2673′ is shown exposed through the gear cover.

An exploded view of the powered finger joint shown in FIG. 26 is shown in FIG. 27. In FIG. 27, the motor 2673 and motor output 2673′ are arranged adjacent to the nested dual-stage cycloidal 2800 so that the long axis through the midline of the motor is parallel to the long axis through the midline of the nested dual-stage cycloidal gear reduction. The motor drives an input gear having a shaft 2885 with an eccentric end region that engages with the first cycloidal pinion 2887 to rotate the cycloidal pinion within the ring gear that is integrally formed with the housing 2886, which in turn drives the output shaft 2888 of this first cycloidal stage 2884. The first output shaft 2888 is also the input shaft to the nested second cycloidal stage 2890 and eccentrically drives a second cycloidal pinion 2891to drive rotation of a floating ring gear 2893 within the cycloidal housing 2894 as the output of the second stage. An output pin 2892 is mounted to the side of the floating ring gear 2893 and may travel in a slot 2895 (output pin channel) in the cycloidal housing 2894. The first end of the powered finger joint may include a cover 2671 covering the gears and the second end of the powered finger joint may include an end cover 2896. Various fasteners 2676 (e.g., screws, pins, etc.) may be used to secure the components together. One or more electrical connectors 2674 may be included and may make electrical connection (e.g., wired connection or in some examples, wireless connection) with additional controllers. These powered finger joints may include one or more sensors, including sensors offering control feedback, such as position sensors, sensors detecting the output (e.g., output pin, etc.), sensors detecting torque, etc. The powered finger joint may also include circuitry, e.g., mounted on printed circuit boards 2675 on an outside or inside of the powered finger joint.

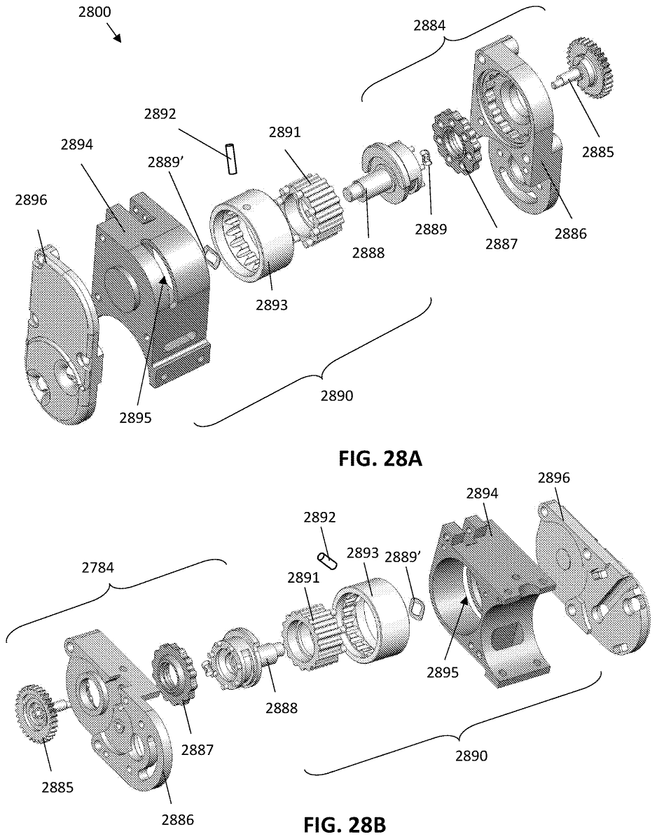

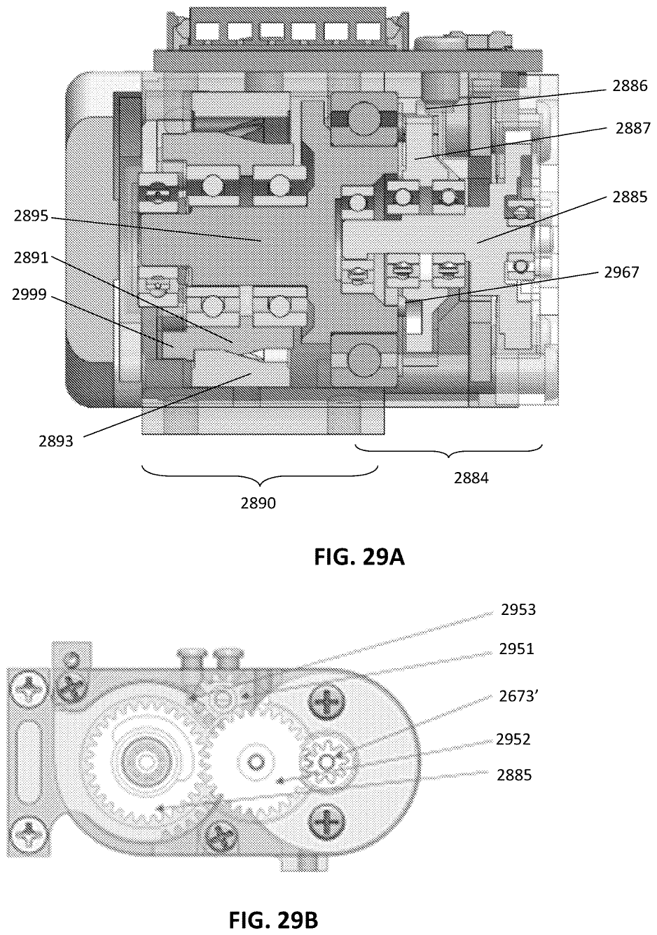

FIGS. 28A-28B show an exploded view of an example of a nested dual-stage cycloidal 2800 of a powered finger joint, showing the arrangement of the elements described above. The first stage output shaft/second stage input shaft 2888 may also include springs (disc springs 2889, 2889′ securing it position. FIGS. 29A-29B, 30A-30B, 31, 32 33, 34, 35 and 36 all illustrate features of powered finger joints such as those described above.

For example, FIG. 29A illustrates a section through one example of a powered finger joint (e.g., finger gear drive unit) having a nested dual-stage cycloidal as described herein. In this example, the finger gear drive unit includes a first cycloidal stage 2884 and a second cycloidal stage 2890. The first cycloidal stage has a first input shaft 2885, an eccentric 14 tooth cycloidal pinion 2887, a 15 tooth cycloidal housing 2886 (ring gear housing) and a pinion-to-input shaft two torque interface 2967. A second stage cycloidal 2890 includes a second input shaft 2895, an eccentric 16 tooth cycloidal pinion 2891, a 17 tooth cycloidal output 2893 and a pinion-to-housing torque interface 2999.

The first and second cycloidal stages are connected together by nesting the end of the first stage drive shaft inside the second stage drive shaft to optimize the space. The torque transfer may be done between the first stage pinion and the second stage drive shaft using an array of pins that allow for the eccentric motion of the pinion but transfers the overall rotation that is the resultant of the 1st stage reduction.

For example, FIG. 29B shows an end view of a finger gear drive unit for transferring rotation of the motor 2673′ to the input gear shaft 2885 of the first cycloidal gear stage. In this example an idler gear 2952 couples the output of the motor to the input of the first cycloidal gear stage. This end of the device also includes within the gear box an output encoder 2951 with a magnet that may be used to determine the relative or absolute position of the joint and the adjacent segment of the finger based on movement of a geared output plate 2953 that include gear teeth that mesh with the output encoder. In any of these apparatuses the same magnetic position sensor that senses the joint angle may also be used to sense joint torque. For example, a flexure spring may convert the joint torque into a linear translation of a second magnet. This magnet may be sensed by a second magnetic sensor that is disposed, e.g., about 1 mm from the magnet and is positioned on a flex circuit PCB. Each joint may include this arrangement. This flexure may be helpful for impedance control which requires the instantaneous joint load sensing so it can compute a dynamics model for the control system.

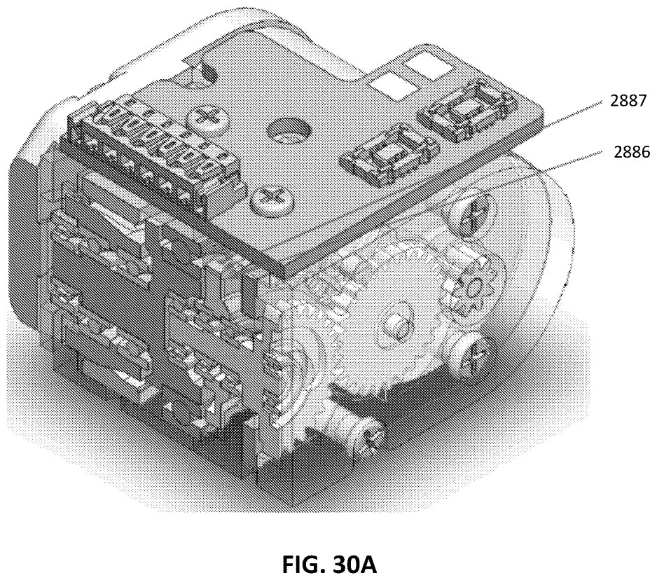

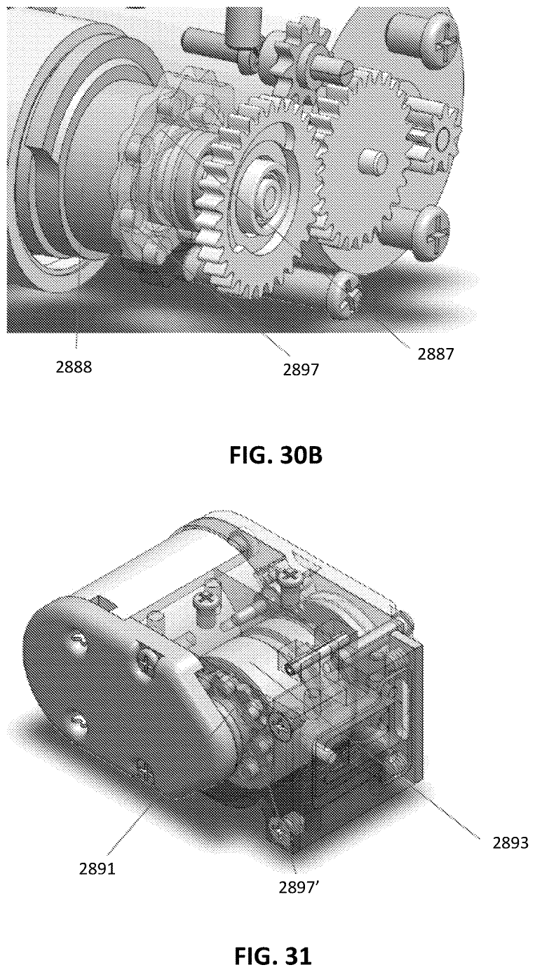

FIG. 30A shows a section through one example of a finger gear drive unit, including an eccentric 14 tooth cycloidal pin 2887 and a 15 tooth cycloidal housing (ring gear housing 2886) as described above. FIG. 30B shows an end view of the finger gear drive unit of FIG. 30A, including the first stage cycloidal pinion 2887, torque transfer pins 2897 and first stage output shaft (second stage input shaft) 2888. As mentioned above, the second stage cycloidal output is inverted where the pinion torque transfer pins are coupled to the stationary housing and the outer ring rotates. As the pinion is driven by the drive shaft, the part is translated in a circular motion, but does not rotate about any axis. This drives the second stage output ring gear to rotate about the central axis of the geartrain. The housing may then be used as a plain bearing interface for the output ring gear, minimizing assembly size, part count, and geartrain length. For example, FIG. 31 shows a perspective view of the finger gear drive unit, including the arrangement of the second stage pinion 2891, torque transfer pins 2897′ and second stage output 2893 (floating ring gear).

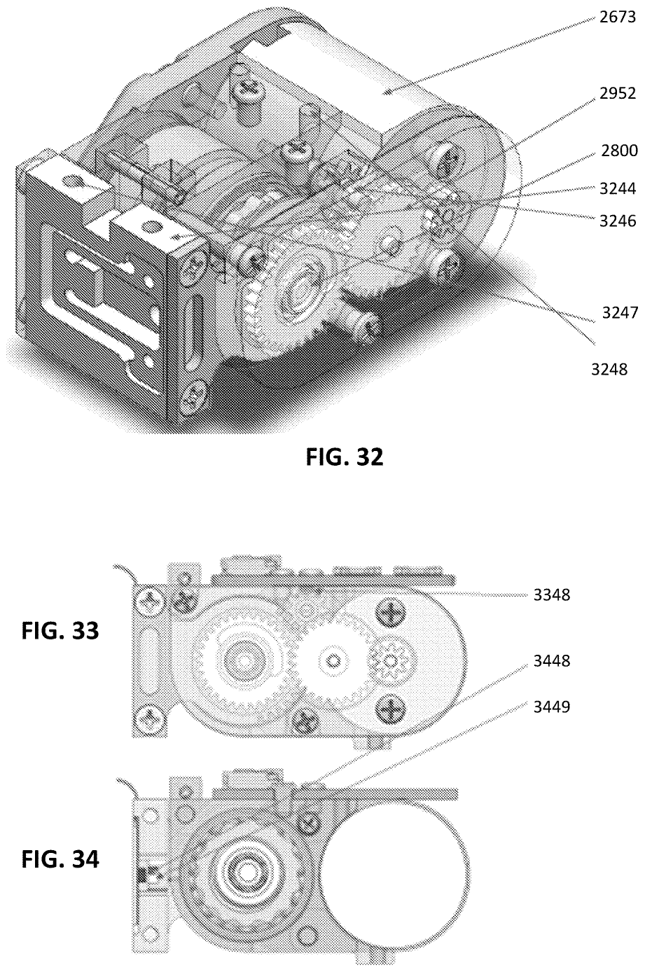

FIG. 32 shows a front perspective view of the finger gear drive unit, including the motor 2673, idler gear 2952, dual-stage cycloidal 2800, and output platform 3244. The apparatus may also include an output position indicator (e.g., diametrically magnetized cylinder) 3246, and may include output platform fastening locations 3247 (e.g., four are shown in this example) and housing fastening locations 3248 (e.g., four are shown in this example).

FIGS. 33 and 34 show front and back views, respectively, of the finger gear drive unit shown in FIGS. 26-34. The output position indicator 3348, which may be a 3D hall effect encoder, is visible in FIG. 33. An output torque encoder 3448 (e.g., a 3D hall effect encoder) and an output torque indicator (e.g., magnet 3449) is visible in FIG. 34.

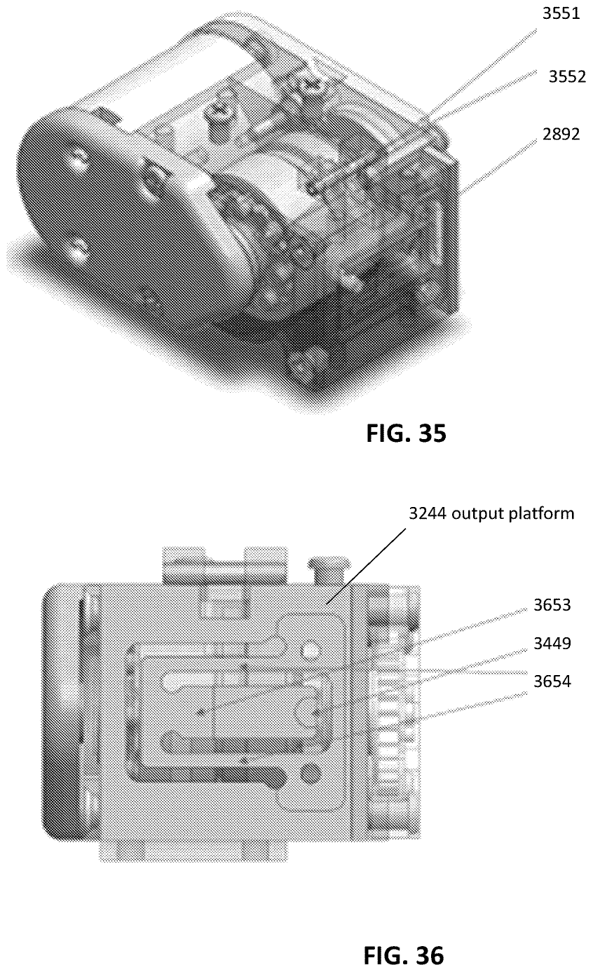

FIG. 35 shows a perspective view of the finger gear drive unit in a partially transparent back perspective view, showing the pinion-to-housing torque interface 3551 (for the second stage), as well as a pivot for the joint linkages 3552. FIG. 35 also shows the output coupling pin 2892. In FIG. 35 the output platform has been made transparent. FIG. 36 shows a side view showing the output platform 3244. The output platform 3244 is connected to the second stage cycloidal output via the output coupling pin (not shown in FIG. 36). The pin is attached to a sprung section on the output platform. When a torque is resisted by the attachment to the output platform, the output will drive the sprung section away from the resting position. Using a Hall effect sensor, a position change of a magnet attached to the spring section of the platform relative to the grounded section may be detected. Knowing the force-displacement relationship of the spring and the distance from the central axis of the geartrain, we can sense torque on the joint. In FIG. 36, the output platform is coupled via an output coupling (behind the output platform in FIG. 36) and one or more flexures may provide a spring compliance. A torque indicator magnet (not shown) may be attached at the spring section of the platform.

A finger gear drive unit may be part of an assembly of multiple segments forming a finger, including multiple finger gear drive units (e.g., forming a finger gear drive assembly) that may include multiple finger gear drive unit interconnected as described herein. This interconnection may be particularly beneficial as it may permit each joint of each finger assembly to operate together in a compact and extremely powerful manner

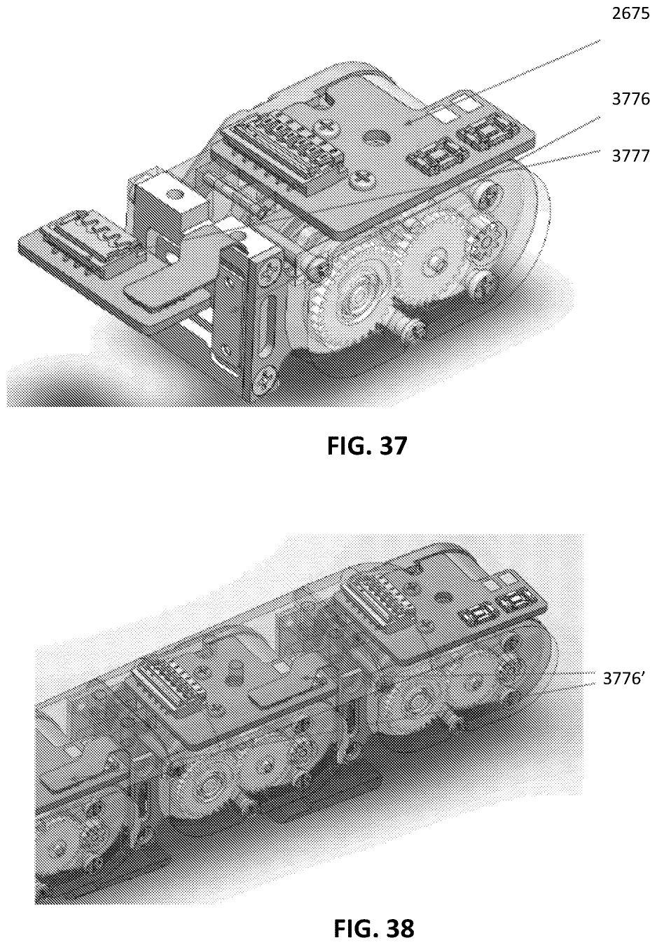

Each finger gear drive unit may include circuitry (e.g., a control board, output encoder board, and/or output encoder communication board) mounted on one or more outer sides of the finger gear drive unit. For example, as shown in FIG. 37, each finger gear drive unit may include an actuator control board 2675 with an output encoder mounted on the upper surface (e.g., a surface that is perpendicular to the output platform). The output encoder board 3776 in this example is shown coupled to the output platform. The apparatus may also include an output encoder communication circuitry (e.g., part of an output encoder communication board 3777).

As shown in FIG. 38, a plurality of finger gear drive units may be coupled together in series. In this example the output encoder board 3776′ of one stage may connect with the actuator control board of the next stage, as shown. This connection may be flexible.

Although the examples described above refer to prosthetic apparatuses, it should be understood that any of these apparatuses may also or alternatively refer to robotic apparatuses. In some examples these apparatuses may be part of a robotic manipulator that is automatically or semi-automatically manipulated.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device.

Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present.

Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-73. (canceled)

74. A powered compact finger joint device, the device comprising:
a motor;
a nested dual-stage cycloidal reduction gear having a first cycloidal stage receiving input from motor, the first cycloidal stage having a first eccentric input shaft, a first cycloidal pinion, and a ring gear housing, wherein the first cycloidal pinion couples to a second cycloidal stage eccentric input shaft, wherein the second cycloidal stage eccentric input shaft couples to a second cycloidal stage pinion driving a floating ring gear within a cycloidal housing; and
an output arm coupled to the floating ring gear and configured to move within an output channel through the cycloidal housing;
wherein the nested dual-stage cycloidal reduction gear is configured to provide a gear reduction of between about 12:1 to about 25:1.

75. The device of claim 74, wherein the motor and the nested dual-stage cycloidal reduction gear are arranged side-by-side and have approximately the same length and same diameter.

76. The device of claim 75, wherein the length is less than 20 mm.

77. The device of claim 74, further comprising a motor output gear coupling the motor to the first eccentric input shaft of the first cycloidal stage.

78. The device of claim 74, wherein the output channel extends laterally around a side of the cycloidal housing.

79. The device of claim 74, wherein the first cycloidal stage is unbalanced.

80. The device of claim 74, further comprising one or more sensor configured to sense a position of the output arm.

81. The device of claim 74, further comprising a sensor configured to detect torque.

82. The device of claim 74, further comprising an output platform pivotally mounted to a lateral side of the device.

83. The device of claim 74, further comprising a control circuitry configured to control operation of the motor.

84. The device of claim 74, wherein the control circuitry is mounted to a lateral side of the device.

85. A robotic finger apparatus comprising, the apparatus comprising:
a pair of powered compact finger joint devices connected in series, wherein each powered compact finger joint device comprises:
a motor;
a nested dual-stage cycloidal reduction gear having a first cycloidal stage receiving input from motor, the first cycloidal stage having a first eccentric input shaft, a first cycloidal pinion, and a ring gear housing, wherein the first cycloidal pinion couples to a second cycloidal stage eccentric input shaft, wherein the second cycloidal stage eccentric input shaft couples to a second cycloidal stage pinion driving a floating ring gear within a cycloidal housing; and
an output arm coupled to the floating ring gear and configured to move within an output channel through the cycloidal housing, wherein the nested dual-stage cycloidal reduction gear is configured to provide a gear reduction of between about 12:1 to about 25:1;
wherein a second of the pair of powered compact finger joint devices is rigidly coupled to an output platform, further wherein the output platform is pivotally coupled to a first of the pair of powered compact finger joint device so that the output arm is drives rotation of the output platform relative to the first powered compact finger joint device.

86. The apparatus of claim 85, further comprising a third powered compact finger joint device coupled to the second of the pair of powered compact finger joint devices through a second output platform, wherein the second output platform is rigidly coupled to the second powered compact finger joint device, further wherein the second output platform is pivotally coupled to the second powered compact finger joint device so that the output arm of the second powered compact finger joint device drives rotation of the second output platform relative to the second powered compact finger joint device.

87. The apparatus of claim 85, wherein the motor and the nested dual-stage cycloidal reduction gear within each powered compact finger joint device are arranged side-by-side and have approximately the same length and same diameter.

88. The apparatus of claim 87, wherein the length is less than 20 mm.

89. The apparatus of claim 85, further comprising a motor output gear within each powered compact finger joint device coupling the motor to the first eccentric input shaft of the first cycloidal stage.

90. The apparatus of claim 85, wherein the output channel within each powered compact finger joint device that extends laterally around a side of the cycloidal housing.

91. The apparatus of claim 85, wherein the first cycloidal stage within each powered compact finger joint device is unbalanced.

92. The apparatus of claim 85, further comprising one or more sensor configured to sense a position of the output arm of each of the powered compact finger joint device.

93. The apparatus of claim 85, further comprising a sensor configured to detect torque between each powered compact finger joint device.

94. The apparatus of claim 85, further comprising a control circuitry on at least one of the powered compact finger joint device configured to control operation of the motor.

95. (canceled)
